SEQUENCE LISTING (95 sequences)

SEQ ID NO: 1 - protein, 514 aa, Homo sapiens
MSWPRRLLLRYLFPALLLHGLGEGSALLHPDSRSHPRSLEKSAWRAFKESQCHHMLKHLHNGARITVQMPPTIEGHWVSTGCEVRSGPEFITRSYRFYHNNTFKAYQFYYGSNRCTNPTYTLIIRGKIRLRQASWIIRGGTEADYQLHNVQVICHTEAVAEKLGQQVNRTCPGFLADGGPWVQDVAYDLWREENGCECTKAVNFAMHELQLIRVEKQYLHHNLDHLVEELFLGDIHTDATQRMFYRPSSYQPPLQNAKNHDHACIACRIIYRSDEHHPPILPPKADLTIGLHGEWVSQRCEVRPEVLFLTRHFIFHDNNNTWEGHYYHYSDPVCKHPTFSIYARGRYSRGVLSSRVMGGTEFVFKVNHMKVTPMDAATASLLNVFNGNECGAEGSWQVGIQQDVTHTNGCVALGIKLPHTEYEIFKMEQDARGRYLLFNGQRPSDGSSPDRPEKRATSYQMPLVQCASSSPRAEDLAEDSGSSLYGRAPGRHTWSLLLAALACLVPLLHWNIRR

SEQ ID NO: 2 - DNA, 22 bp, synthetic construct
  source 1..22: mol_type = other DNA
catatctccatcgcctcagttg

SEQ ID NO: 3 - DNA, 19 bp, synthetic construct
  source 1..19: mol_type = other DNA
ggcaggagtccatgactgt

SEQ ID NO: 4 - DNA, 20 bp, synthetic construct
  source 1..20: mol_type = other DNA
cgtggcttactccccattta

SEQ ID NO: 5 - DNA, 20 bp, synthetic construct
  source 1..20: mol_type = other DNA
tctcgctgtctctccctctc

SEQ ID NO: 6 - DNA, 20 bp, synthetic construct
  source 1..20: mol_type = other DNA
cctcctgctcctcctttctt

SEQ ID NO: 7 - DNA, 20 bp, synthetic construct
  source 1..20: mol_type = other DNA
gacgcagacgatgtatgcac

SEQ ID NO: 8 - DNA, 22 bp, synthetic construct
  source 1..22: mol_type = other DNA
ggtacattccacattcgtgagc

SEQ ID NO: 9 - DNA, 22 bp, synthetic construct
  source 1..22: mol_type = other DNA
tcacgtgcaataatcaaaacca

SEQ ID NO: 10 - DNA, 22 bp, synthetic construct
  source 1..22: mol_type = other DNA
acagcgctgcagaatgccaagt

SEQ ID NO: 11 - DNA, 22 bp, synthetic construct
  source 1..22: mol_type = other DNA
gaagtggacctcacgctggtgc

SEQ ID NO: 12 - DNA, 20 bp, synthetic construct
  source 1..20: mol_type = other DNA
gggcagggtggctccaggat

SEQ ID NO: 13 - DNA, 20 bp, synthetic construct
  source 1..20: mol_type = other DNA
tgctgaccgggacggaggag

SEQ ID NO: 14 - DNA, 20 bp, synthetic construct
  source 1..20: mol_type = other DNA
ccgttctccatcaacaacct

SEQ ID NO: 15 - DNA, 20 bp, synthetic construct
  source 1..20: mol_type = other DNA
ggggtagtgcatcacctgtt

SEQ ID NO: 16 - DNA, 20 bp, synthetic construct
  source 1..20: mol_type = other DNA
ttgaggtcaatgaaggggtc

SEQ ID NO: 17 - DNA, 20 bp, synthetic construct
  source 1..20: mol_type = other DNA
gaaggtgaaggtcggagtca

SEQ ID NO: 18 - DNA, 21 bp, synthetic construct
  source 1..21: mol_type = other DNA
gttcccgccgtcgctgatgat

SEQ ID NO: 19 - DNA, 22 bp, synthetic construct
  source 1..22: mol_type = other DNA
gccggtgtagacgaaatggccg

SEQ ID NO: 20 - DNA, 22 bp, synthetic construct
  source 1..22: mol_type = other DNA
gacgctgctcaactccatggca

SEQ ID NO: 21 - DNA, 22 bp, synthetic construct
  source 1..22: mol_type = other DNA
ttggcggcgtcgatgtagagga

SEQ ID NO: 22 - DNA, 20 bp, synthetic construct
  source 1..20: mol_type = other DNA
gtacggctacctgggtcaac

SEQ ID NO: 23 - DNA, 20 bp, synthetic construct
  source 1..20: mol_type = other DNA
acttgggtctcgttgagctg

SEQ ID NO: 24 - DNA, 20 bp, synthetic construct
  source 1..20: mol_type = other DNA
cgtcgctcgctgagtgcctg

SEQ ID NO: 25 - DNA, 23 bp, synthetic construct
  source 1..23: mol_type = other DNA
tgtcgagtgtgaaagcgtcgagg

SEQ ID NO: 26 - DNA, 20 bp, synthetic construct
  source 1..20: mol_type = other DNA
ggccaatctgctgaacctca

SEQ ID NO: 27 - DNA, 20 bp, synthetic construct
  source 1..20: mol_type = other DNA
gagttcagatagccaccggc

SEQ ID NO: 28 - DNA, 22 bp, synthetic construct
  source 1..22: mol_type = other DNA
tcaacggcatctacgagttcat

SEQ ID NO: 29 - DNA, 22 bp, synthetic construct
  source 1..22: mol_type = other DNA
aagcacttgttgagggacagat

SEQ ID NO: 30 - DNA, 22 bp, synthetic construct
  source 1..22: mol_type = other DNA
ggccttctcagtactaccctct

SEQ ID NO: 31 - DNA, 22 bp, synthetic construct
  source 1..22: mol_type = other DNA
ccgtagctcgagggatgaaaat

SEQ ID NO: 32 - DNA, 22 bp, synthetic construct
  source 1..22: mol_type = other DNA
ggcttgcgccccgtagaaatgt

SEQ ID NO: 33 - DNA, 22 bp, synthetic construct
  source 1..22: mol_type = other DNA
aggagccaggtcaggtccgaac

SEQ ID NO: 34 - DNA, 21 bp, synthetic construct
  source 1..21: mol_type = other DNA
gggcgaccacttcggcatgaa

SEQ ID NO: 35 - DNA, 22 bp, synthetic construct
  source 1..22: mol_type = other DNA
cgtcggcatggttgaagtgtgc

SEQ ID NO: 36 - DNA, 20 bp, synthetic construct
  source 1..20: mol_type = other DNA
cgcatcgtttcttctcctct

SEQ ID NO: 37 - DNA, 20 bp, synthetic construct
  source 1..20: mol_type = other DNA
cagacagacttggggctcac

SEQ ID NO: 38 - DNA, 20 bp, synthetic construct
  source 1..20: mol_type = other DNA
agggcggggcacagattgga

SEQ ID NO: 39 - DNA, 20 bp, synthetic construct
  source 1..20: mol_type = other DNA
gctggcagagtgtgcccaga

SEQ ID NO: 40 - DNA, 23 bp, synthetic construct
  source 1..23: mol_type = other DNA
ggatcccaactcggacgacgaga

SEQ ID NO: 41 - DNA, 21 bp, synthetic construct
  source 1..21: mol_type = other DNA
aggatgagctctccggctcgg

SEQ ID NO: 42 - DNA, 20 bp, synthetic construct
  source 1..20: mol_type = other DNA
acaagtggccaattcactcc

SEQ ID NO: 43 - DNA, 20 bp, synthetic construct
  source 1..20: mol_type = other DNA
gaggtggacaagggatctga

SEQ ID NO: 44 - DNA, 22 bp, synthetic construct
  source 1..22: mol_type = other DNA
ccccattgtgacaggccgtgac

SEQ ID NO: 45 - DNA, 22 bp, synthetic construct
  source 1..22: mol_type = other DNA
tcagcgtcggtgctgagtagct

SEQ ID NO: 46 - DNA, 20 bp, synthetic construct
  source 1..20: mol_type = other DNA
tggtattctctccccctcct

SEQ ID NO: 47 - DNA, 20 bp, synthetic construct
  source 1..20: mol_type = other DNA
taaggatgttgaacgggcag

SEQ ID NO: 48 - DNA, 20 bp, synthetic construct
  source 1..20: mol_type = other DNA
cttcagtgggaggtcaggtt

SEQ ID NO: 49 - DNA, 18 bp, synthetic construct
  source 1..18: mol_type = other DNA
caaacacagcatcgacgg

SEQ ID NO: 50 - DNA, 22 bp, synthetic construct
  source 1..22: mol_type = other DNA
atagctgccgactaagcattga

SEQ ID NO: 51 - DNA, 19 bp, synthetic construct
  source 1..19: mol_type = other DNA
atccgtgcgaaggtgcttt

SEQ ID NO: 52 - DNA, 20 bp, synthetic construct
  source 1..20: mol_type = other DNA
ccaattacaaccccgacatc

SEQ ID NO: 53 - DNA, 20 bp, synthetic construct
  source 1..20: mol_type = other DNA
agtttcactcctggccactg

SEQ ID NO: 54 - DNA, 20 bp, synthetic construct
  source 1..20: mol_type = other DNA
accggcctcactcccacaca

SEQ ID NO: 55 - DNA, 20 bp, synthetic construct
  source 1..20: mol_type = other DNA
cgctcggtccaatggcctgg

SEQ ID NO: 56 - DNA, 22 bp, synthetic construct
  source 1..22: mol_type = other DNA
ctcaacaagaatgagtcggtgc

SEQ ID NO: 57 - DNA, 22 bp, synthetic construct
  source 1..22: mol_type = other DNA
actccttggtgaacttgtggtt

SEQ ID NO: 58 - DNA, 20 bp, synthetic construct
  source 1..20: mol_type = other DNA
ctttcttgtgctgcatacgg

SEQ ID NO: 59 - DNA, 18 bp, synthetic construct
  source 1..18: mol_type = other DNA
agctcagcaagacgctgg

SEQ ID NO: 60 - DNA, 20 bp, synthetic construct
  source 1..20: mol_type = other DNA
tcgtccccgctcaagagcga

SEQ ID NO: 61 - DNA, 22 bp, synthetic construct
  source 1..22: mol_type = other DNA
ccttggcgcagttcttctcgca

SEQ ID NO: 62 - DNA, 20 bp, synthetic construct
  source 1..20: mol_type = other DNA
gagccacgagtttggatgtt

SEQ ID NO: 63 - DNA, 20 bp, synthetic construct
  source 1..20: mol_type = other DNA
tgcagggagaaaggagagaa

SEQ ID NO: 64 - DNA, 22 bp, synthetic construct
  source 1..22: mol_type = other DNA
gcgatggccccactcggatact

SEQ ID NO: 65 - DNA, 22 bp, synthetic construct
  source 1..22: mol_type = other DNA
tagctgcccagagcctgcttca

SEQ ID NO: 66 - DNA, 22 bp, synthetic construct
  source 1..22: mol_type = other DNA
atgtggccgaggactttgattg

SEQ ID NO: 67 - DNA, 22 bp, synthetic construct
  source 1..22: mol_type = other DNA
atggcaagggacttcctgtaac

SEQ ID NO: 68 - DNA, 22 bp, synthetic construct
  source 1..22: mol_type = other DNA
acaaggaacagaacggagtaca

SEQ ID NO: 69 - DNA, 22 bp, synthetic construct
  source 1..22: mol_type = other DNA
gtatgaggaaaccttgggctgt

SEQ ID NO: 70 - DNA, 22 bp, synthetic construct
  source 1..22: mol_type = other DNA
gtctgctcttctgcctcttgat

SEQ ID NO: 71 - DNA, 22 bp, synthetic construct
  source 1..22: mol_type = other DNA
tacgtcaagtcggcaaggtatg

SEQ ID NO: 72 - DNA, 22 bp, synthetic construct
  source 1..22: mol_type = other DNA
ggagggcttttaaggagtcaca

SEQ ID NO: 73 - DNA, 22 bp, synthetic construct
  source 1..22: mol_type = other DNA
cactgtgatccttgcaccattg

SEQ ID NO: 74 - DNA, 22 bp, synthetic construct
  source 1..22: mol_type = other DNA
tggaggcacaaaacactactga

SEQ ID NO: 75 - DNA, 22 bp, synthetic construct
  source 1..22: mol_type = other DNA
tggggacagtggacttgtttag

SEQ ID NO: 76 - DNA, 22 bp, synthetic construct
  source 1..22: mol_type = other DNA
cacggctacagagtatcttccc

SEQ ID NO: 77 - DNA, 22 bp, synthetic construct
  source 1..22: mol_type = other DNA
ggggaggtaactgggagaaaag

SEQ ID NO: 78 - DNA, 20 bp, synthetic construct
  source 1..20: mol_type = other DNA
gtcacgggagtaccagaacg

SEQ ID NO: 79 - DNA, 23 bp, synthetic construct
  source 1..23: mol_type = other DNA
tgtagacaaagtactgggcatcg

SEQ ID NO: 80 - DNA, 22 bp, synthetic construct
  source 1..22: mol_type = other DNA
ctctgaagacctactgggagga

SEQ ID NO: 81 - DNA, 22 bp, synthetic construct
  source 1..22: mol_type = other DNA
gcattcgcaattgggttacctt

SEQ ID NO: 82 - DNA, 22 bp, synthetic construct
  source 1..22: mol_type = other DNA
catttctacttccccacaccca

SEQ ID NO: 83 - DNA, 22 bp, synthetic construct
  source 1..22: mol_type = other DNA
ggctgtagttgctgaccttgta

SEQ ID NO: 84 - DNA, 22 bp, synthetic construct
  source 1..22: mol_type = other DNA
caacaatctccagacactccca

SEQ ID NO: 85 - DNA, 22 bp, synthetic construct
  source 1..22: mol_type = other DNA
cccctcaggtccacatttgtta

SEQ ID NO: 86 - DNA, 22 bp, synthetic construct
  source 1..22: mol_type = other DNA
ggaaggtggaagaaaaagagcg

SEQ ID NO: 87 - DNA, 22 bp, synthetic construct
  source 1..22: mol_type = other DNA
ggaaacacatcagccctactca

SEQ ID NO: 88 - DNA, 22 bp, synthetic construct
  source 1..22: mol_type = other DNA
tcggaccctaaggctgttttac

SEQ ID NO: 89 - DNA, 22 bp, synthetic construct
  source 1..22: mol_type = other DNA
ctttcaggggcaggaagaagat

SEQ ID NO: 90 - DNA, 22 bp, synthetic construct
  source 1..22: mol_type = other DNA
gctacccaaataaccagctcca

SEQ ID NO: 91 - DNA, 22 bp, synthetic construct
  source 1..22: mol_type = other DNA
cgatcctcacatgggtgaaagt

SEQ ID NO: 92 - DNA, 22 bp, synthetic construct
  source 1..22: mol_type = other DNA
tctggagcctgatgtcttaacg

SEQ ID NO: 93 - DNA, 22 bp, synthetic construct
  source 1..22: mol_type = other DNA
gacgcagcagaaataaagcaca

SEQ ID NO: 94 - DNA, 22 bp, synthetic construct
  source 1..22: mol_type = other DNA
aataattcgctggtgagcctga

SEQ ID NO: 95 - DNA, 22 bp, synthetic construct
  source 1..22: mol_type = other DNA
ccaggtggaggctttctagatg

(19) United States
(12) Patent Application Publication
(10) Pub. No.: US 2026/0266818 A1
(43) Pub. Date: Sep. 10, 2026

(54) BIOLOGICAL METHODS

(71) Applicant: Somite Therapeutics, Inc., Boston, MA (US)

(72) Inventors: Agnete Kirkeby, Hellerup (DK); Alrik Schoerling, Lund (SE)

(21) Appl. No.: 19/165,622

(22) PCT Filed: Mar. 15, 2024
(86) PCT No.: PCT/EP2024/056926

§ 371 Date: Sep. 16, 2025

(30) Foreign Application Priority Data
Mar. 17, 2023 (EP) 23162638.3

(51) Int. Cl. G01N 33/569; A61K 35/30; G01N 33/58

(52) U.S. Cl. CPC G01N 33/56966; A61K 35/30; G01N 33/582

(57) ABSTRACT

The invention relates to a method of identifying one or more caudal ventral midbrain (cVM) progenitor cells by measuring the presence and/or amount of adenomatosis polyposis coli down-regulated 1 (APCDD1) expressed by the one or more cells. The invention also provides methods of enriching cVM cells, uses of said cells in the treatment of disease, use of APCDD1 as a biomarker, and kits.

The invention relates to a method of identifying one or more caudal ventral midbrain (cVM) progenitor cells by measuring the presence and/or amount of adenomatosis polyposis coli down-regulated 1 (APCDD1) expressed by the one or more cells. The invention also provides methods of enriching cVM cells, uses of said cells in the treatment of disease, use of APCDD1 as a biomarker, and kits.

Parkinson's disease (PD) is common neurodegenerative movement disorder. Levodopa is the most common treatment strategy for ameliorating the motor symptoms of this disease, but is associated with serious complications and the efficiency fluctuates and progressively diminishes (Connolly and Lang 2014). Since the motor symptoms of PD can be attributed to a relatively selective and focal loss of dopaminergic (DA) neurons within the ventral midbrain (VM), cell replacement is a promising alternative treatment strategy to levodopa.

The feasibility of cell replacement therapy has been demonstrated by using fetal VM-derived cells (Barker et al. 2019; Björklund et al. 1980; Bolam et al. 1987; Dunnett et al. 1983; Freund et al. 1985; Strecker et al. 1987). Due to tissue-related issues however, the focus has recently shifted towards using human pluripotent stem cells (hPSCs) as a near inexhaustible source of VM DA cells (Kefalopoulou et al. 2014; Kurowska et al. 2011; J. Y. Li et al. 2008; W. Li et al. 2016). The cells are transplanted as progenitors that mature in the host brain to replace the functions of endogenously lost cells.

hPSCs are a promising source of caudal ventral midbrain (cVM) progenitor cells that are used in cell replacement therapy for Parkinson's disease. However, some heterogeneity in cell composition is inevitable in the cell differentiation process. Cell surface markers predictive of functional maturation of cVM progenitors to ventral midbrain (VM) dopaminergic (DA) neurons could be used as a convenient quality control and to enrich for the target cell type. However, until the present invention, the use of such markers has not been successful in providing a bona fide cVM progenitor marker.

A pure population of cells must be transplanted for safe, efficacious, and reproducible outcome. Genes indicative of VM DA progenitors, such as the transcription factors LMX1A, FOXA2 and OTX2, are commonly used surrogate markers for functional maturation (Arenas, Denham, and Villaescusa 2015). These markers are incorporated in intracellularly based flow cytometric quality control (QC) panels that are assessing the purity of the differentiated cells. However, only caudal VM (cVM)-derived LMX1A/FOXA2/OTX2 triple-positive progenitors give rise to VM DA neurons whereas triple-positive cells of the rostrally-adjacent subthalamic nucleus (STN), referred to as the rostral VM (rVM) from this point, produces glutamatergic neurons (Nolbrant et al. 2017). Current flow cytometry-based QC assays using LMX1A/FOXA2/OTX2 are unable to distinguish between cVM and rVM fates. Indeed, FOXA2, OTX2 and LMX1A expression levels fail to predict graft outcome upon transplantation (Kirkeby et al. 2017).

There are several published, putative surface markers predictive of VM DA neurons (Bye et al. 2015; Doi et al. 2014; Fathi et al. 2018; Gennet et al. 2016; Kikuchi et al. 2017; Lehnen et al. 2017; Paik et al. 2018; Samata et al. 2016; Sundberg et al. 2013; Yoo et al. 2021), some of which ameliorate behavioural deficits in Parkinsonian animal models (Doi et al. 2014; Kikuchi et al. 2017; Lehnen et al. 2017; Sundberg et al. 2013; Yoo et al. 2021). Apart from the surface marker CORIN, few of these have been extensively characterized, and the regional specificity is underexplored.

Whereas several of these studies have focused on the enrichment of LMX1A+ and FOXA2+ cells from a mixed starting progenitor population, few have focused on benchmarking the specificity of the markers against a range of other regional neural progenitor cells to scrutinize the specificity of the markers against potentially contaminating populations of other neural fates. In addition, there has been little focus on assessing the specificity of the published markers for the true DA progenitor cells of the cVM, which are LMX1A+FOXA2+EN1+ versus the neighbouring non-DA rVM progenitor, which are LMX1A+FOXA2+EN1− (Kee et al. 2017; Kirkeby et al. 2017).

The inventors therefore identified a need to identify markers truly enriched for a bona fide VM DA fate to aid the development of accurate and efficient QC assays of cell products that will be used in phase-I clinical trials exploring cell replacement therapy as a PD treatment.

Herein, the inventors have identified a new cell surface marker of cVM progenitors—adenomatosis polyposis coli down-regulated 1 (APCDD1). The inventors have compared the performance of this marker to seven previously published cVM progenitor surface markers. The markers were benchmarked for specificity in the cVM, the sensitivity to different dissociation enzymes, and reproducibility.

The inventors have showed that out of the tested surface markers, APCDD1 and trophoblast glycoprotein (TPBG) were the most enriched in cVM progenitors compared to neural progenitor cells from other neighbouring regions in the brain. APCDD1 has been shown to have a higher reproducibility than TPBG, rendering it more useful as a quality control marker. APCDD1 correlated highly to markers of a cVM fate. Sorting of APCDD1+ cells enriched for cVM genes while depleting contaminating cell populations. Transplanted APCDD1+ cells, sorted from mixed cells, but not APCDD1− cells, yielded full behavioural amelioration in Parkinsonian rat model. Animals with APCDD1+-derived grafts had an increased in bona fide VM DA neurons compared to APCDD1− grafts.

Taken together, the inventors have shown that APCDD1 outperforms previously published surface markers, and will be useful in identifying cVM progenitor cells for quality control of cell products used in upcoming clinical trials.

A first aspect of the invention provides a method of identifying one or more caudal ventral midbrain (cVM) progenitor cells comprising or consisting of the steps of:
(i) providing one or more cells;
(ii) measuring the presence and/or amount of adenomatosis polyposis coli down-regulated 1 (APCDD1) expressed by the one or more cells;
   wherein the presence and/or amount of APCDD1 expressed by the one or more cells is indicative of the one or more cells being cVM progenitor cells.

In human central nervous system development, as part of neurulation, the neural plate rolls up to form the neural tube. Forward to back, the neural tube is comprised of the forebrain (telencephalon and diencephalon), midbrain (mesencephalon) and hindbrain (rhombencephalon). Between the midbrain and hindbrain sits the midbrain-hindbrain boundary. The bottom of the midbrain is the ventral midbrain (VM) and the back of the VM is the caudal VM (cVM), situated just rostrally (forward) of the midbrain-hindbrain boundary. The caudal VM (cVM) can be discriminated from the non-dopaminergic rostral VM (rVM) through the expression of EN1. Only cVM progenitor cells give rise to VM dopaminergic neurons belonging to the Substantia Nigra of the human brain. A hallmark of Parkinson's disease is the relatively selective loss of VM dopaminergic neurons in the Substantia Nigra, which causes the motor symptoms of the disease.

By “cVM progenitor cells” we include any cell on the differentiation pathway from a stem cell to a mature cVM cell or a ventral midbrain dopaminergic neuron. Identification of cVM progenitor cells has been challenging in the field, as so far no cell surface marker has been identified allowing reliable discrimination of cVM progenitor cells from other midbrain progenitor cell fates. The present invention solves this problem by identifying the marker APCDD1, which is shown herein to be specific to cVM progenitor cells.

By “providing one or more cells” we include any cell or cell population from which cVM progenitor cells are to be identified. This may take the form of a population of cells that have been differentiated into the cVM progenitor fate, e.g. from a starting population of stem cells or cells derived from fetal tissue. The one or more cells may also be a single cell that is suspected of being a cVM progenitor cell.

By “measuring the presence and/or amount of APCDD1 expressed by the one or more cells” we include carrying out any method to detect or measure APCDD1 expression by the one or more cells. In some embodiments, the expression of APCDD1 is on the surface of the one or more cells. In some embodiments, the expression of APCDD1 is within the one or more cells. The term also includes measurement of the presence and/or amount of either APCDD1 protein or the corresponding pre-mRNA or mature mRNA. In some preferred embodiments, the method involves measuring the presence of APCDD1 expressed by the one or more cells.

By “APCDD1” we refer to Adenomatosis polyposis coli down-regulated 1 protein (also known as DRAPC1, B7323 or FP7019). APCDD1 is a single-pass type I membrane protein that has been shown previously to be expressed in the gastrointestinal tract, placental trophoblasts, and both epidermal and dermal compartments from human hair follicles.

In some embodiments, the APCDD1 is mammalian APCDD1. In some preferred embodiments, the APCDD1 is human APCDD1. In some embodiments, the APCDD1 has the amino acid sequence shown in UniProt entry Q8J025 (human APCDD1), shown here as SEQ ID NO: 1:

(SEQ ID NO: 1)
MSWPRRLLLRYLFPALLLHGLGEGSALLHPDSRSHPRSLEKSAWRA
FKESQCHHMLKHLHNGARITVQMPPTIEGHWVSTGCEVRSGPEFI
TRSYRFYHNNTFKAYQFYYGSNRCTNPTYTLIIRGKIRLRQASWI
IRGGTEADYQLHNVQVICHTEAVAEKLGQQVNRTCPGFLADGGPW
VQDVAYDLWREENGCECTKAVNFAMHELQLIRVEKQYLHHNLDHL
VEELFLGDIHTDATQRMFYRPSSYQPPLQNAKNHDHACIACRIIY
RSDEHHPPILPPKADLTIGLHGEWVSQRCEVRPEVLFLTRHFIFH
DNNNTWEGHYYHYSDPVCKHPTFSIYARGRYSRGVLSSRVMGGTE
FVFKVNHMKVTPMDAATASLLNVFNGNECGAEGSWQVGIQQDVTH
TNGCVALGIKLPHTEYEIFKMEQDARGRYLLFNGQRPSDGSSPDR
PEKRATSYQMPLVQCASSSPRAEDLAEDSGSSLYGRAPGRHTWSL
LLAALACLVPLLHWNIRR

By “APCDD1” we refer to both the APCDD1 protein, and the corresponding mRNA expressed within the one or more cells. In some preferred embodiments, APCDD1 refers to APCDD1 protein. In some preferred embodiments, APCDD1 refers to APCDD1 protein expressed on the surface of the one or more cells. Therefore, in some preferred embodiments, the method involves measuring the presence and/or amount of APCDD1 expressed on the surface of the one or more cells.

In the claimed method, the one or more cells are identified as expressing APCDD1 and can then be categorised as cVM progenitor cells based on this expression. This categorisation is useful for enriching populations of cVM progenitor cells, for applications such as cell replacement therapy, where there is a need for highly pure populations of cVM progenitor cells.

Therefore, in some embodiments, the method is for enriching a population of cVM progenitor cells, and wherein the method further comprises:
(iii) isolating the one or more cells that are identified as cVM progenitor cells to obtain a cVM progenitor cell or population enriched for cVM progenitor cells.

By “enriching a population of cVM progenitor cells” we include a method of obtaining a population of cells comprising a higher proportion of cVM progenitor cells than the starting population of one or more cells. “Enriching” may be used interchangeably with “purifying” in the art.

In some embodiments, isolating the one or more cells involves selecting for the cVM progenitor cells in the starting population, thereby excluding other contaminating cell types. In some other embodiments, this may involve actively removing contaminating cell types from the population of one or more cells, leaving an enriched population of cVM progenitor cells.

In some embodiments, the invention provides a method of enriching a population of cVM progenitor cells, and wherein the method comprises:
(i) providing one or more cells;
(ii) measuring the presence and/or amount of adenomatosis polyposis coli down-regulated 1 (APCDD1) expressed by the one or more cells; and
(iii) isolating the one or more cells that are identified as cVM progenitor cells to obtain a cVM progenitor cell or population enriched for cVM progenitor cells.

In some embodiments, the one or more cells provided in step (i) are neural progenitor cells. In some embodiments, the one or more cells provided in step (i) are a population comprising neural progenitor cells.

Neural progenitor cells are the progenitor cells of the central nervous system (CNS) that give rise to mature cells of the CNS. In some particular embodiments, the neural progenitor cells may be progenitor cells of the midbrain. In some particular embodiments, the neural progenitor cells may be selected from the group comprising: dorsal forebrain (dFB), ventral forebrain (vFB), dorsal midbrain (dMB), rostral ventral midbrain (rVM), dorsal hindbrain (dHB) and ventral hindbrain (vHB) progenitor cells.

In some embodiments, the population comprising neural progenitor cells comprises a mixture of different types of neural progenitor cells, for example those described in the preceding paragraph.

In some embodiments, the one or more cells provided in step (i) are neural progenitor cells that have been obtained from fetal tissue or differentiated from stem cells. In some embodiments, the stem cells are induced pluripotent stem cells (iPSC) or embryonic stem cells (ES).

In some embodiments, the fetal tissue comprises fetal stem cells from which the one or more cells in step (i) can be derived or differentiated. In some embodiments, the fetal tissue comprises fetal neural stem cells. In this embodiment, the one or more cells in step (i) can be differentiated from fetal neural stem cells, for example to form one or more neural progenitor cells from which cVM progenitor cells.

In some other embodiments, one or more neural progenitor cells can be obtained directly from fetal tissue.

In some preferred embodiments, the one or more cells provided in step (i) are cells that have been differentiated from iPSCs, for example are neural progenitor cells differentiated from iPSCs. In some preferred embodiments, the iPSCs are human iPSCs (hiPSCs).

By “iPSC” we mean any pluripotent stem cells that has been obtained by reprogramming of a somatic cell. By “ES cell” we mean any pluripotent stem cells that have been derived from embryonic tissue. For the purposes of the present application, it will be understood that ES cells used in the claimed methods can be derived from parthenogenetically activated human oocytes without leading to the destruction of a human embryo.

In some preferred embodiments, the fetal tissue, iPSCs and ES cells described herein are mammalian tissue/cells. In some preferred embodiments, the fetal tissue, iPSCs and ES cells described herein are human tissue/cells.

By “differentiated” we mean that the stem cells described herein have been directed using external factors to form a particular type of cell, e.g. a cVM progenitor cell. In some embodiments, this is termed directed differentiation. The skilled person will be aware that directing differentiation is normally achieved by treating stem cells with particular growth factors and/or cytokines and/or caudalising and/or ventralising factors. The skilled person will be aware of the methods of directing differentiation of cVM progenitor cells.

Therefore, in some embodiments, the one or more cells in step (i) may comprise any neural progenitor cells derived from fetal tissue or differentiated from stem cells. This may be any one of the neural progenitor cell types discussed herein, mature neural cells (for example mature rVM cells), or undifferentiated cells derived from fetal tissue or stem cells.

In some embodiments, the method is for quality control during preparation of cells for cell replacement therapy.

In some embodiments, the method is for providing cVM progenitor cells for cell replacement therapy.

In some embodiments, the method is for determining the suitability of cells for use in treatment, optionally wherein the treatment is cell replacement therapy.

Cell replacement therapy involves the administration or transplant of cells (or the appropriate progenitor cells) to allow the replacement of dead, damaged, or non-functional cells with healthy, functional cells. For example, in the context of the present application, the cVM progenitor cells identified or enriched using the methods of the invention may be transplanted or administered to the brain of a patient in order to replace lost or non-functional dopamine cells.

Cell replacement therapy is proposed to be an important treatment for Parkinson's disease, as it has the ability to replace dead or dying dopaminergic neurons, thereby restoring lost function and minimising symptoms. cVM progenitor cells can be administered to a patient, which then differentiate in situ into mature dopaminergic neurons.

It is important to ensure any transplanted cVM progenitor cells are highly pure and homogeneous to maximise the effect of the procedure, and ensure there are no unwanted side effects. However, when differentiating stem cells to cVM progenitor cells, other neural progenitor cell types are often formed and some stem cells remain undifferentiated. These contaminating cell populations can compromise the cells for transplant. It is therefore important to ensure a highly pure population of cVM cells are obtained, and this has previously been difficult to achieve when enriching populations of cVM progenitors based on cell surface markers. This process of ensuring a highly pure population of cVM progenitors have been obtained is referred to as quality control.

However, the present invention solves this problem by presenting a new marker APCDD1 that can successfully discriminate between cVM progenitors and contaminating cells types. Importantly, APCDD1 can discriminate between cVM progenitor cells and the similar rVM progenitor cells with high efficiency.

This makes APCDD1 particularly suitable as a marker for quality control of cVM progenitor cell production and enrichment.

In some embodiments, step (ii) comprises measuring the presence or amount of APCDD1 expression on the surface of one or more cells.

APCDD1 is a cell surface protein, and therefore in some preferred embodiments, the methods of the invention involve detecting expression of APCDD1 protein on the surface of cells. The advantage of this method is it allows APCDD1 measurement on intact cells without the need for lysing.

In some embodiments, step (ii) comprises measuring the presence of APCDD1. In some preferred embodiments, step (ii) comprises measuring the presence of APCDD1 using an antibody based detection method, for example flow cytometry. In some embodiments, step (ii) comprises any method that is capable of determining whether or not a cell is positive for APCDD1. By “positive for APCDD1”, we mean that the cell has measurable expression of APCDD1.

In some embodiments, measuring the presence and/or amount of APCDD1 is performed using a first binding moiety specific for APCDD1.

In some embodiments, the measurement is performed using a second binding moiety specific for the first binding moiety. For example, if the first binding moiety is an antibody, the second binding moiety may be a further antibody or antigen binding fragment thereof specific for the Fc portion of the first binding moiety.

In some embodiments, the first and/or second binding moiety is an antibody or antigen binding fragment thereof, or a variant thereof.

Methods for the production and use of antibodies are well known in the art, for example see Antibodies: A Laboratory Manual, 1988, Harlow & Lane, Cold Spring Harbor Press, ISBN-13: 978-0879693145, Using Antibodies: A Laboratory Manual, 1998, Harlow & Lane, Cold Spring Harbor Press, ISBN-13: 978-0879695446 and Making and Using Antibodies: A Practical Handbook, 2006, Howard & Kaser, CRC Press, ISBN-13: 978-0849335280 (the disclosures of which are incorporated herein by reference).

Thus, a fragment may contain one or more of the variable heavy (V_{H}) or variable light (V_{L}) domains. For example, the term antibody fragment includes Fab-like molecules (Better et al (1988) Science 240, 1041); Fv molecules (Skerra et al (1988) Science 240, 1038); single-chain Fv (scFv) molecules where the V_{H} and V_{L} partner domains are linked via a flexible oligopeptide (Bird et al (1988) Science 242, 423; Huston et al (1988) Proc. Natl. Acad. Sci. USA 85, 5879) and single domain antibodies (dAbs) comprising isolated V domains (Ward et al (1989) Nature 341, 544).

For example, the binding moieties may be scFv molecules. In some preferred embodiments, the binding moieties are whole antibodies (i.e. comprising both the Fc and Fv).

The term “antibody variant” includes any synthetic antibodies, recombinant antibodies or antibody hybrids, such as but not limited to, a single-chain antibody molecule produced by phage-display of immunoglobulin light and/or heavy chain variable and/or constant regions, or other immunointeractive molecule capable of binding to an antigen in an immunoassay format that is known to those skilled in the art.

A general review of the techniques involved in the synthesis of antibody fragments which retain their specific binding sites is to be found in Winter & Milstein (1991) Nature 349, 293-299.

Molecular libraries such as antibody libraries (Clackson et al, 1991, Nature 352, 624-628; Marks et al, 1991, J Mol Biol 222(3): 581-97), peptide libraries (Smith, 1985, Science 228(4705): 1315-7), expressed cDNA libraries (Santi et al (2000) J Mol Biol 296(2): 497-508), libraries on other scaffolds than the antibody framework such as affibodies (Gunneriusson et al, 1999, Appl Environ Microbiol 65(9): 4134-40) or libraries based on aptamers (Kenan et al, 1999, Methods Mol Biol 118, 217-31) may be used as a source from which binding moieties that are specific for a given motif are selected for use in the methods of the invention.

In one embodiment of the methods of the invention, step (ii) is performed using an assay comprising a first binding moiety capable of binding to APCDD1. The first binding moiety may also comprise a detectable moiety. In some alternative embodiments, the first binding moiety does not comprise a detectable moiety and a second binding moiety comprising a detectable moiety that is specific for the first binding moiety is used.

The second binding moiety may be as described above in relation to the (first) binding moiety, such as an antibody or antigen-binding fragment thereof.

Alternatively, or in addition, the first and/or second binding moieties may be labelled with a detectable moiety.

By a “detectable moiety” we include the meaning that the moiety is one which may be detected and the relative amount and/or location of the moiety (for example, the location on an array) determined.

Suitable detectable moieties are well known in the art. For example, the detectable moiety may be selected from the group consisting of: a fluorescent moiety; a luminescent moiety; a chemiluminescent moiety; a radioactive moiety; an enzymatic moiety.

The detectable moiety may be a fluorescent and/or luminescent and/or chemiluminescent moiety which, when exposed to specific conditions, may be detected. For example, a fluorescent moiety may need to be exposed to radiation (i.e., light) at a specific wavelength and intensity to cause excitation of the fluorescent moiety, thereby enabling it to emit detectable fluorescence at a specific wavelength that may be detected.

Alternatively, the detectable moiety may be an enzyme which is capable of converting a (preferably undetectable) substrate into a detectable product that can be visualised and/or detected.

In a further alternative, the detectable moiety may be a radioactive atom which is useful in imaging. Suitable radioactive atoms include ^{99m}Tc and ¹²³I for scintigraphic studies.

Other readily detectable moieties include, for example, spin labels for magnetic resonance imaging (MRI) such as ¹²³I again, ¹³¹I, ¹¹¹In, ¹⁹F, ¹³C, ¹⁵N, ¹⁷O, gadolinium, manganese or iron. Clearly, the agent to be detected (such as, for example, the one or more biomarkers in the test sample and/or control sample described herein and/or an antibody molecule for use in detecting a selected protein) must have sufficient of the appropriate atomic isotopes in order for the detectable moiety to be readily detectable.

In some preferred embodiments, the first and/or second binding moiety is fluorescently labelled. Suitable fluorescent probes are well known in the art, and include but are not limited to: CF568; Alexa Fluor 488, 568, 405, 647 and 700; R-PE; PerCP-Cy5.5; R-PE-Cy7; APC.

In some other embodiments, the first and/or second binding moiety is magnetically labelled. Suitable magnetic probes are well known in the art and include paramagnetic nanoparticles.

In some embodiments, the measurement of the presence and/or amount of APCDD1 is performed by flow cytometry, qRT-PCR, RNA sequencing or other RNA-detecting methods, immunostaining, chromatography.

In some preferred embodiments, the measurement of the presence and/or amount of APCDD1 is performed by flow cytometry specific for APCDD1.

By “flow cytometry” we include methods that use laser light to sort stained or labelled samples of cells based on their labelling/staining profile. For example, in the case of APCDD1 expressing cells, flow cytometry can be used to detect and isolate cells labelled with an APCDD1 specific antibody.

In some embodiments, measuring the presence and/or amount of APCDD1 and/or isolation of cells expressing APCDD1 is performed by flow cytometry, optionally wherein the flow cytometry is Fluorescence Activated Cell Sorting (FACS) or Magnetic Affinity Cell Sorting (MACS).

In some embodiments, when step (ii) is performed by FACS, the binding moieties are fluorescently labelled as discussed herein. In some other embodiments, when step (ii) is performed by MACS, the binding moieties are magnetically labelled as discussed herein.

In some other embodiments, APCDD1 is detected using qRT-PCR, RNA sequencing or another RNA detection method. Therefore, in these embodiments, it is the APCDD1 specific mRNA that is detected as opposed to protein.

In some other embodiments, APCDD1 is detected using immunostaining. By “immunostaining” we mean the process of identifying cells expressing APCDD1 by treating a sample with a binding moiety specific for APCDD1 and then visualising the identified cells, e.g. by using a labelled primary antibody or a secondary labelled antibody. The antibodies may be labelled with fluorescent labels described herein. Immunostaining may be a particularly useful technique if cVM progenitor cells are to be isolated from a tissue sample, e.g. a fetal tissue sample.

In some other embodiments, APCDD1 is detected using chromatography. By “chromatography” we mean any technique that relies on separation of materials in a mixture based on their properties, e.g. whether a cell binds to an anti-APCDD1 binding moiety or not. Therefore, step (ii) of the method may therefore involve the use of affinity chromatography based methods.

It will be understood that a skilled person may combine any of the techniques described herein in order to carry out step (ii) of the method. For example, a crude first step may involve immunostaining or chromatography, followed by a more refined second step of flow cytometry, leading to a highly pure sample of cVM progenitor cells.

In some embodiments, the method further comprises administering the identified cVM progenitor cells into the brain of a subject, preferably wherein the subject is a human.

By “brain” we include the cerebrum, cerebellum, and brainstem. In some preferred embodiments, the cVM progenitor cells are administered directly to the putamen, which is the target site for midbrain dopamine neurons in humans.

In some embodiments, the cVM progenitor cells are administered to the part of the brain of the subject where target projections derived from cVM progenitor cells are usually found. In some embodiments, the cVM progenitor cells are administered directly to the forebrain. In some embodiments, the cVM progenitor cells are administered directly to the basal ganglia area of the forebrain. In some embodiments, the cVM progenitor cells are administered directly to the putamen area of the forebrain. In some embodiments, the cVM progenitor cells are administered directly into the substantia nigra pars compacta.

In some embodiments, the method is for the treatment of a neurological disease or condition. In some preferred embodiments, the neurological disease or condition is Parkinson's disease. In some embodiments, the neurological disease or condition is another other Parkinsonian condition. By “Parkinsonian condition” we mean any disorder or syndrome that shares clinical features with Parkinson's disease, but has a different pathology.

Parkinson's disease (PD) is a degenerative neurological condition that is characterised by cell death in the basal ganglia of the brain, specifically death of dopaminergic neurons in the midbrain (e.g. in the caudal ventral midbrain). Cell death is typically caused by overexpression and/or misfolding of the protein alpha-synuclein, which aggregates and causes cell death. PD is characterised by the following symptoms: tremor, bradykinesia, rigidity, shuffling/stooped gait, autonomic dysfunction (dysautonomia), neuropsychiatric problems (mood, cognition, behaviour or thought alterations), and sensory (e.g. altered sense of smell) and sleep difficulties.

In some embodiments, the subject has diagnosed Parkinson's disease. In some other embodiments, the subject may have suspected Parkinson's disease. In some other embodiments, the subject may have early-stage Parkinson's disease. In some other embodiments, the subject may have late-stage Parkinson's disease. In some other embodiments, the subject may have another Parkinsonian condition.

In some embodiments, the method produces a substantially enriched population of cVM progenitor cells. In some embodiments, the method produces a substantially pure population of cVM progenitor cells.

By “substantially enriched” or “substantially pure” we mean that the population of cVM progenitor cells is highly pure and contains very few contaminating cell types. In some embodiments, this means that the population of cVM progenitor cells contains a substantially higher proportion of cVM progenitor cells following carrying out the claimed method.

In some embodiments, the substantially enriched/pure population comprises undetectable levels of cell types other than cVM progenitor cells. For example, these cell types other than cVM progenitor cells may not be detectable by flow cytometry.

In some embodiments, the cVM progenitor cells identified by the method are capable of differentiating into ventral midbrain dopaminergic neurons.

In some embodiments, the ventral midbrain dopaminergic neurons express tyrosine hydroxylase (TH).

Ventral midbrain dopaminergic neurons are key for controlling movement, processing and memory. These neurons are responsible for producing dopamine, and if this process is disrupted it can lead to loss of function, for example as seen in Parkinson's disease. These neurons are often associated with the expression of TH, as this enzyme is essential in the conversion of tyrosine to dopamine. A ventral midbrain dopaminergic neuron is therefore any neuronal cell in the ventral midbrain that produces or is capable of producing dopamine.

In some embodiments of the method, the cVM progenitor cells may be further characterised according to expression of one or more known markers that were previously known to be associated with cVM progenitor cells. However, none of these previously identified markers were bona fide markers of cVM progenitor cells, as they are also expressed on other neural progenitor cells, e.g. rVM progenitor cells. For example, previous QC assays for cVM progenitor cells relied on detection of LMX1A, FOXA2, and OTX2, however these three markers are also markers of rVM cells which give rise to glutaminergic neurons instead of dopaminergic neurons.

In some embodiments, the cVM progenitor cells identified by the method also express one or more of the following markers: FOXA2; OTX2; LMX1A and EN1.

The inventors have also found herein that the combination of APCDD1 positive and CORIN negative cells could discriminate between cVM progenitor cells and rVM progenitor cells with the highest efficiency.

Therefore, in some preferred embodiments, the cVM progenitor cells identified by the method do not express CORIN. In some preferred embodiments, the method further comprises measuring the presence/absence and/or amount of CORIN.

In some embodiments, the cVM progenitor cells identified by the method do not express CORIN.

In some embodiments, the cVM progenitor cells also express FOXA2 and/or OTX2, and the method further comprises: measuring the presence and/or amount of FOXA2 and/or OTX2 expression in the one or more cells; and wherein the presence and/or amount of APCDD1 and FOXA2 and/or OTX2 on or in the one or more cells is indicative of the one or more cells being cVM progenitor cells.

In some embodiments, the cVM progenitor cells also express FOXA2, and the method further comprises: measuring the presence and/or amount of FOXA2 expression in the one or more cells; and wherein the presence and/or amount of APCDD1 and FOXA2 on or in the one or more cells is indicative of the one or more cells being cVM progenitor cells.

In some embodiments, the cVM progenitor cells also express OTX2, and the method further comprises: measuring the presence and/or amount of OTX2 expression in the one or more cells; and wherein the presence and/or amount of APCDD1 and OTX2 on or in the one or more cells is indicative of the one or more cells being cVM progenitor cells.

In some embodiments, the cVM progenitor cells do not express CORIN, and the method further comprises: measuring the presence and/or amount of CORIN on the surface of the one or more cells; and wherein the presence and/or amount of APCDD1 expression on the one or more cells and the absence and/or amount of CORIN expression on the one or more cells is indicative of the one or more cells being cVM progenitor cells.

In some preferred embodiments, the cVM progenitor cells do not express CORIN, and the method further comprises: measuring the presence and/or amount of CORIN on the surface of the one or more cells; and wherein the presence and/or amount of APCDD1 expression on the one or more cells and the absence and/or amount of CORIN expression on the one or more cells is indicative of the one or more cells being cVM progenitor cells.

In some embodiments, the cVM progenitor cells have one of the following marker profiles:
(a) APCDD1 positive and CORIN negative;
(b) APCDD1 positive, FOXA2 positive, and OTX2 positive;
(c) APCDD1 positive, CORIN negative, FOXA2 positive, and OTX2 positive.

In some preferred embodiments, the cVM progenitor cells are APCDD1 positive and CORIN negative. In this embodiment, the method described herein is able to discriminate between cVM and rVM progenitor cells with high efficiency.

In some embodiments, the method is capable of discriminating between cVM progenitor cells and neural progenitor cells or between cVM progenitor cells and neural cells of one or more of the following lineages: dorsal forebrain (dFB), ventral forebrain (vFB), dorsal midbrain (dMB), rostral ventral midbrain (rVM), dorsal hindbrain (dHB) and ventral hindbrain (vHB).

In some preferred embodiments, the method is capable of discriminating between cVM progenitor cells and rVM progenitor cells. In some preferred embodiments, the method is capable of discriminating between cVM progenitor cells and rVM cells.

A second aspect of the invention provides a cell or population of cells produced according to the method of the invention.

In some embodiments, this population of cells produced according to the method of the invention is a highly enriched or pure population of cVM progenitor cells.

A third aspect of the invention provides a population of substantially enriched population of cVM progenitor cells produced according to the method of the invention.

A fourth aspect of the invention provides an isolated cell or isolated population of cells that are cVM progenitor cells, wherein the cells express APCDD1 on the surface.

In some embodiments, the cell or population of cells are for use in a method of treating a neurological condition or disease. In some preferred embodiments, the neurological condition or disease is Parkinson's disease.

A fifth aspect of the invention provides a cVM progenitor cell or population thereof for use in a method of treating a neurological condition or disease in a subject in need thereof, wherein the method comprises carrying out the method of the invention discussed herein, and administering a therapeutically effective amount of the cVM cells identified to the subject.

A sixth aspect of the invention provides use of a cVM progenitor cell or population thereof for the manufacture of a medicament for the treatment of a neurological condition or disease in a subject in need thereof.

A seventh aspect of the invention provides a method of treating a subject having a neurological condition or disease comprising administering to a subject a therapeutically effective amount of the cell or population of cells described herein.

The uses and methods of treatment described herein preferably involve administration of the cVM progenitor cells as part of a pharmaceutical composition.

In some preferred embodiments, the neurological condition or disease is Parkinson's disease.

In some embodiments, the use or method comprises administering the cell or population thereof into the brain of the subject.

The invention also provides a pharmaceutical composition comprising the cell or population of cVM progenitor cells described herein and a pharmaceutically acceptable carrier or excipient.

In some embodiments, the cVM progenitor cells are administered to the part of the brain of the subject where cells derived from cVM progenitor cells are usually found. In some embodiments, the cVM progenitor cells are administered directly to the forebrain. In some embodiments, the cVM progenitor cells are administered directly to the basal ganglia area of the forebrain. In some embodiments, the cVM progenitor cells are administered directly to the putamen area of the forebrain. In some embodiments, the cVM progenitor cells are administered directly into the substantia nigra pars compacta.

The pharmaceutical compositions may be prepared in a manner known in the art that is sufficiently storage stable and suitable for administration to humans and animals. For example, the pharmaceutical compositions may be lyophilised, e.g. through freeze drying, spray drying, spray cooling, or through use of particle formation from supercritical particle formation.

By “pharmaceutically acceptable” we mean a non-toxic material that does not decrease the effectiveness of the cVM progenitor cells. Such pharmaceutically acceptable buffers, carriers or excipients are well-known in the art (see Remington's Pharmaceutical Sciences, 18th edition, A. R Gennaro, Ed., Mack Publishing Company (1990) and handbook of Pharmaceutical Excipients, 3rd edition, A. Kibbe, Ed., Pharmaceutical Press (2000), the disclosures of which are incorporated by reference).

The term “buffer” is intended to mean an aqueous solution containing an acid-base mixture with the purpose of stabilising pH. Examples of buffers are Trizma, Bicine, Tricine, MOPS, MOPSO, MOBS, Tris, Hepes, HEPBS, MES, phosphate, carbonate, acetate, citrate, glycolate, lactate, borate, ACES, ADA, tartrate, AMP, AMPD, AMPSO, BES, CABS, cacodylate, CHES, DIPSO, EPPS, ethanolamine, glycine, HEPPSO, imidazole, imidazoleacetic acid, PIPES, SSC, SSPE, POPSO, TAPS, TABS, TAPSO and TES.

The term “diluent” is intended to mean an aqueous or non-aqueous solution with the purpose of diluting the agent in the pharmaceutical preparation. The diluent may be one or more of saline, water, polyethylene glycol, propylene glycol, ethanol or oils (such as safflower oil, corn oil, peanut oil, cottonseed oil or sesame oil).

The term “adjuvant” is intended to mean any compound added to the formulation to increase the biological effect of the agent of the invention. The adjuvant may be one or more of zinc, copper or silver salts with different anions, for example, but not limited to fluoride, chloride, bromide, iodide, thiocyanate, sulfite, hydroxide, phosphate, carbonate, lactate, glycolate, citrate, borate, tartrate, and acetates of different acyl composition. The adjuvant may also be cationic polymers such as cationic cellulose ethers, cationic cellulose esters, deacetylated hyaluronic acid, chitosan, cationic dendrimers, cationic synthetic polymers such as poly(vinyl imidazole), and cationic polypeptides such as polyhistidine, polylysine, polyarginine, and peptides containing these amino acids.

The excipient may be one or more of carbohydrates, polymers, lipids and minerals. Examples of carbohydrates include lactose, glucose, sucrose, mannitol, and cyclodextrines, which are added to the composition, e.g. for facilitating lyophilisation. Examples of polymers are starch, cellulose ethers, cellulose carboxymethylcellulose, hydroxypropylmethyl cellulose, hydroxyethyl cellulose, ethylhydroxyethyl cellulose, alginates, carrageenans, hyaluronic acid and derivatives thereof, polyacrylic acid, polysulphonate, polyethylene glycol/polyethylene oxide, polyethylene oxide/polypropylene oxide copolymers, polyvinylalcohol/polyvinylacetate of different degree of hydrolysis, and polyvinylpyrrolidone, all of different molecular weight, which are added to the composition, e.g. for viscosity control, for achieving bioadhesion, or for protecting the lipid from chemical and proteolytic degradation. Examples of lipids are fatty acids, phospholipids, mono-, di-, and triglycerides, ceramides, sphingolipids and glycolipids, all of the different acyl chain length and saturation, egg lecithin, soy lecithin, hydrogenated egg and soy lecithin, which are added to the composition for reasons similar to those for polymers. Examples of minerals are talc, magnesium oxide, zinc oxide and titanium oxide, which are added to the composition to obtain benefits such as reduction of liquid accumulation or advantageous pigment properties.

The agents of the invention may be formulated into any type of pharmaceutical composition known in the art to be suitable for the delivery thereof.

In one embodiment, the pharmaceutical compositions of the invention may be in the form of a liposome, in which the agent is combined, in addition to other pharmaceutically acceptable carriers, with amphipathic agents such as lipids, which exist in aggregated forms as micelles, insoluble monolayers and liquid crystals. Suitable lipids for liposomal formulation include, without limitation, monoglycerides, diglycerides, sulfatides, lysolecithin, phospholipids, saponin, bile acids, and the like. Suitable lipids also include the lipids above modified by poly(ethylene glycol) in the polar headgroup for prolonging bloodstream circulation time. Preparation of such liposomal formulations can be found in for example U.S. Pat. No. 4,235,871, the disclosures of which are incorporated herein by reference.

The pharmaceutical compositions of the invention may also be in the form of biodegradable microspheres. Aliphatic polyesters, such as poly(lactic acid) (PLA), poly(glycolic acid) (PGA), copolymers of PLA and PGA (PLGA) or poly(caprolactone) (PCL), and polyanhydrides have been widely used as biodegradable polymers in the production of microspheres. Preparations of such microspheres can be found in U.S. Pat. No. 5,851,451 and in EP 0 213 303, the disclosures of which are incorporated herein by reference.

In a further embodiment, the pharmaceutical compositions of the invention are provided in the form of polymer gels, where polymers such as starch, cellulose ethers, cellulose carboxymethylcellulose, hydroxypropylmethyl cellulose, hydroxyethyl cellulose, ethylhydroxyethyl cellulose, alginates, carrageenans, hyaluronic acid and derivatives thereof, polyacrylic acid, polyvinyl imidazole, polysulphonate, polyethylene glycol/polyethylene oxide, polyethylene oxide/polypropylene oxide copolymers, polyvinylalcohol/polyvinylacetate of different degree of hydrolysis, and polyvinylpyrrolidone are used for thickening of the solution containing the agent. The polymers may also comprise gelatin or collagen.

Alternatively, the agents may simply be dissolved in saline, water, polyethylene glycol, propylene glycol, ethanol or oils (such as safflower oil, corn oil, peanut oil, cottonseed oil or sesame oil), tragacanth gum, and/or various buffers.

It will be appreciated that the pharmaceutical compositions of the invention may include ions and a defined pH for potentiation of action of the active agent. Additionally, the compositions may be subjected to conventional pharmaceutical operations such as sterilisation and/or may contain conventional adjuvants such as preservatives, stabilisers, wetting agents, emulsifiers, buffers, fillers, etc.

The pharmaceutical compositions according to the invention may be administered via any suitable route known to those skilled in the art.

In some preferred embodiments, the compositions are administered by injection of the composition into the desired area of the brain (e.g. during brain surgery). In some embodiments, the compositions are administered by injection into the putamen area of the forebrain.

Formulations suitable for administration include aqueous and non-aqueous sterile injection solutions which may contain anti-oxidants, buffers, bacteriostats and solutes which render the formulation isotonic with the CSF of the intended recipient; and aqueous and non-aqueous sterile suspensions which may include suspending agents and thickening agents. The formulations may be presented in unit-dose or multi-dose containers, for example sealed ampoules and vials, and may be stored in a cryopreserved condition requiring the addition of the sterile liquid carrier, for example water for injections, immediately prior to use. Extemporaneous injection solutions and suspensions may be prepared from sterile powders, granules and tablets of the kind previously described.

The pharmaceutical compositions will be administered to a patient in a pharmaceutically effective dose. A ‘therapeutically effective amount’, or ‘effective amount’, or ‘therapeutically effective’, as used herein, refers to that amount which provides a therapeutic effect for a given condition and administration regimen. This is a predetermined quantity of active material calculated to produce a desired therapeutic effect in association with the required additive and diluent, i.e. a carrier or administration vehicle. Further, it is intended to mean an amount sufficient to reduce and most preferably prevent, a clinically significant deficit in the activity, function and response of the host. Alternatively, a therapeutically effective amount is sufficient to cause an improvement in a clinically significant condition in a host. As is appreciated by those skilled in the art, the amount of a compound may vary depending on its specific activity. Suitable dosage amounts may contain a predetermined quantity of active composition calculated to produce the desired therapeutic effect in association with the required diluent. In the methods and use for manufacture of compositions of the invention, a therapeutically effective amount of the active component is provided. A therapeutically effective amount can be determined by the ordinary skilled medical or veterinary worker based on patient characteristics, such as age, weight, sex, condition, complications, other diseases, etc., as is well known in the art. The administration of the pharmaceutically effective dose can be carried out both by single administration in the form of an individual dose unit or else several smaller dose units and also by multiple administrations of subdivided doses at specific intervals. Alternatively, the does may be provided as a continuous infusion over a prolonged period.

The compositions of the invention can be formulated at various concentrations, depending on the efficacy/toxicity of the compound being used. The skilled person will be aware of suitable techniques for determining formulations and dosages to be used in practice in administration to patients.

It will be appreciated by persons skilled in the art that the pharmaceutical compositions of the invention may be administered alone or in combination with other therapeutic agents used in the treatment of a neurological disease or condition. In particular, the therapeutic agent(s) may be ones known to be effective to the indication of interest, for example, Parkinson's disease (e.g. levodopa).

In some embodiments, the subject has diagnosed Parkinson's disease. In some other embodiments, the subject may have suspected Parkinson's disease. In some other embodiments, the subject may have early-stage Parkinson's disease. In some other embodiments, the subject may have late-stage Parkinson's disease.

The cVM progenitor cells may be administered in a single dose, or in multiple doses in order to achieve the desired therapeutic effect.

Successful treatment of Parkinson's disease as described herein may involve the improvement (to any degree) or complete alleviation of Parkinson's disease symptoms for any period of time. The improvement may be short term, or more preferable, long term. In some embodiments, the improvement may be permanent.

An eighth aspect of the invention provides use of APCDD1 as a biomarker for identifying and/or enriching for cVM progenitor cells from a population comprising one or more cells.

A ninth aspect of the invention provides use of a binding moiety specific for APCDD1 for identifying and/or enriching for cVM progenitor cells from a population of one or more cells.

A tenth aspect of the invention provides a kit comprising:
(i) a binding moiety specific for APCDD1, optionally wherein the binding moiety is labelled;
(ii) wash and blocking buffer;
(iii) positive and negative control(s);
(iv) optionally, instructions for use.

In some embodiments, the binding moiety is an antibody or an antigen-binding fragment thereof as described herein.

Embodiments of the invention are described in the following numbered paragraphs:
1. A method of identifying one or more caudal ventral midbrain (cVM) progenitor cells comprising or consisting of the steps of:
   (i) providing one or more cells;
   (ii) measuring the presence and/or amount of adenomatosis polyposis coli down-regulated 1 (APCDD1) expressed by the one or more cells;
wherein the presence and/or amount of APCDD1 expressed by the one or more cells is indicative of the one or more cells being cVM progenitor cells.
2. The method of paragraph 1, wherein the method is for enriching a population of cVM progenitor cells, and wherein the method further comprises:
   (iii) isolating the one or more cells that are identified as cVM progenitor cells to obtain a cVM progenitor cell or population enriched for cVM progenitor cells.
3. The method of any one of the preceding paragraphs, wherein the one or more cells provided in step (i) are neural progenitor cells.
4. The method of any one of the preceding paragraphs, wherein the one or more cells provided in step (i) are neural progenitor cells that have been obtained from fetal tissue or differentiated from stem cells, optionally wherein the stem cells are induced pluripotent stem cells (iPSC) or embryonic stem cells (ES).
5. The method of any one of the preceding paragraphs, wherein the method is for quality control during preparation of cells for cell replacement therapy.
6. The method of any one of the preceding paragraphs, wherein the method is for providing cVM progenitor cells for cell replacement therapy.
7. The method of any one of the preceding paragraphs, wherein the method is for determining the suitability of cells for use in treatment, optionally wherein the treatment is cell replacement therapy.
8. The method of any one of the preceding paragraphs, wherein step (ii) comprises measuring the presence or amount of APCDD1 expression on the surface of one or more cells.
9. The method of any one of the preceding paragraphs, wherein step (ii) comprises measuring the presence of APCDD1.
10. The method of any one of the preceding paragraphs, wherein measuring the presence and/or amount of APCDD1 is performed using a first binding moiety specific for APCDD1.
11. The method of paragraph 10, wherein the measurement is performed using a second binding moiety specific for the first binding moiety.
12. The method of paragraphs 10 or 11 wherein the first and/or second binding moiety is an antibody or antigen binding fragment thereof.
13. The method of any of paragraphs 10-12 wherein the first and/or second binding moiety is fluorescently labelled.
14. The method of any one of the preceding paragraphs, wherein the measurement of the presence and/or amount of APCDD1 is performed by flow cytometry, qRT-PCR, RNA sequencing or other RNA-detecting methods, immunostaining, chromatography.
15. The method of any one of paragraph 14, wherein measuring the presence and/or amount of APCDD1 and/or isolation of cells expressing APCDD1 is performed by flow cytometry, optionally wherein the flow cytometry is Fluorescence Activated Cell Sorting (FACS) or Magnetic Affinity Cell Sorting (MACS).
16. The method of any one of the preceding paragraphs, wherein the method further comprises administering the identified cVM progenitor cells into the brain of a subject, preferably wherein the subject is a human.
17. The method of paragraph 16, wherein the method is for the treatment of a neurological disease or condition, optionally wherein the neurological disease or condition is Parkinson's disease.
18. The method of any one of the preceding paragraphs, wherein the method produces a substantially enriched population of cVM progenitor cells.
19. The method of any one of the preceding paragraphs, wherein the cVM progenitor cells identified by the method are capable of differentiating into ventral midbrain dopaminergic neurons.
20. The method of paragraph 19, wherein the ventral midbrain dopaminergic neurons express tyrosine hydroxylase (TH).
21. The method of any one of the preceding paragraphs, wherein the cVM progenitor cells identified by the method also express one or more of the following markers: FOXA2; OTX2; LMX1A and EN1.
22. The method of any one of the preceding paragraphs, wherein the cVM progenitor cells identified by the method do not express CORIN.
23. The method of any one of the preceding paragraphs, wherein the cVM progenitor cells also express FOXA2 and/or OTX2, and the method further comprises: measuring the presence and/or amount of FOXA2 and/or OTX2 expression in the one or more cells; and wherein the presence and/or amount of APCDD1 and FOXA2 and/or OTX2 on or in the one or more cells is indicative of the one or more cells being cVM progenitor cells.
24. The method of any one of the preceding paragraphs, wherein the cVM progenitor cells do not express CORIN, and the method further comprises: measuring the presence and/or amount of CORIN on the surface of the one or more cells; and wherein the presence and/or amount of APCDD1 expression on the one or more cells and the absence and/or amount of CORIN expression on the one or more cells is indicative of the one or more cells being cVM progenitor cells.
25. The method of any one of the preceding paragraphs, wherein the cVM progenitor cells have one of the following marker profiles:
   (a) APCDD1 positive and CORIN negative;
   (b) APCDD1 positive, FOXA2 positive, and OTX2 positive;
   (c) APCDD1 positive, CORIN negative, FOXA2 positive, and OTX2 positive.
26. The method of any one of the preceding paragraphs, wherein the method is capable of discriminating between cVM progenitor cells and neural progenitor cells or between cVM progenitor cells and neural cells of one or more of the following lineages: dorsal forebrain (dFB), ventral forebrain (vFB), dorsal midbrain (dMB), rostral ventral midbrain (rVM), dorsal hindbrain (dHB) and ventral hindbrain (vHB), preferably wherein the method is capable of discriminating between cVM progenitor cells and rVM progenitor cells or rVM cells.
27. A cell or population of cells produced according to the method of paragraphs 1-26.
28. A population of substantially enriched population of cVM progenitor cells produced according to the method of paragraphs 1-26.
29. An isolated cell or isolated population of cells that are cVM progenitor cells, wherein the cells express APCDD1 on the surface.
30. The cell or population of cells of paragraphs 27-29 for use in a method of treating a neurological condition or disease.
31. A cVM progenitor cell or population thereof for use in a method of treating a neurological condition or disease in a subject in need thereof, wherein the method comprises carrying out the method of any one of paragraphs 1-26, and administering a therapeutically effective amount of the cVM cells identified to the subject.
32. Use of a cVM progenitor cell or population thereof of paragraphs 27-29 for the manufacture of a medicament for the treatment of a neurological condition or disease in a subject in need thereof.
33. A method of treating a subject having a neurological condition or disease comprising administering to a subject a therapeutically effective amount of the cell or population of cells of paragraphs 27-29.
34. The cell or population of cells for use of paragraphs 30-31, the use of paragraph 32, or the method of paragraph 33, wherein the neurological condition or disease is Parkinson's disease.
35. The cell or population of cells for use of paragraphs 30-31, the use of paragraph 32, or the method of paragraph 33, wherein the use or method comprises administering the cell or population thereof into the brain of the subject.
36. Use of APCDD1 as a biomarker for identifying and/or enriching for cVM progenitor cells from a population comprising one or more cells.
37. Use of a binding moiety specific for APCDD1 for identifying and/or enriching for cVM progenitor cells from a population of one or more cells.
38. A kit comprising:
   (i) a binding moiety specific for APCDD1, optionally wherein the binding moiety is labelled;
   (ii) wash and blocking buffer;
   (iii) positive and negative control(s);
   (iv) optionally, instructions for use.
39. The use of paragraph 37 or the kit of paragraph 38, wherein the binding moiety is an antibody or an antigen-binding fragment thereof.

Preferred, non-limiting examples which embody certain aspects of the invention will now be described, with reference to the following figures:

### DESCRIPTION OF THE FIGURES

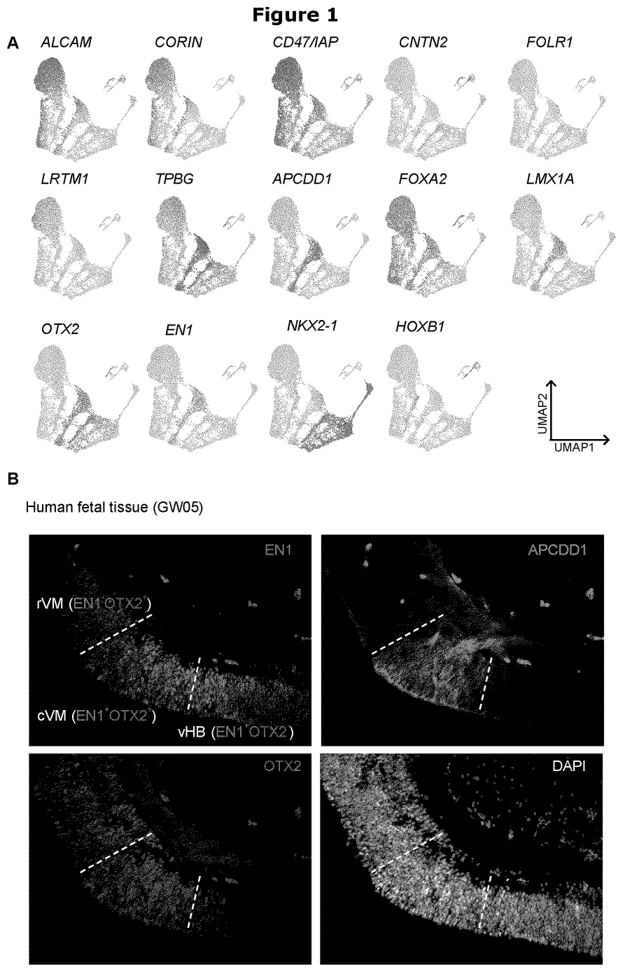

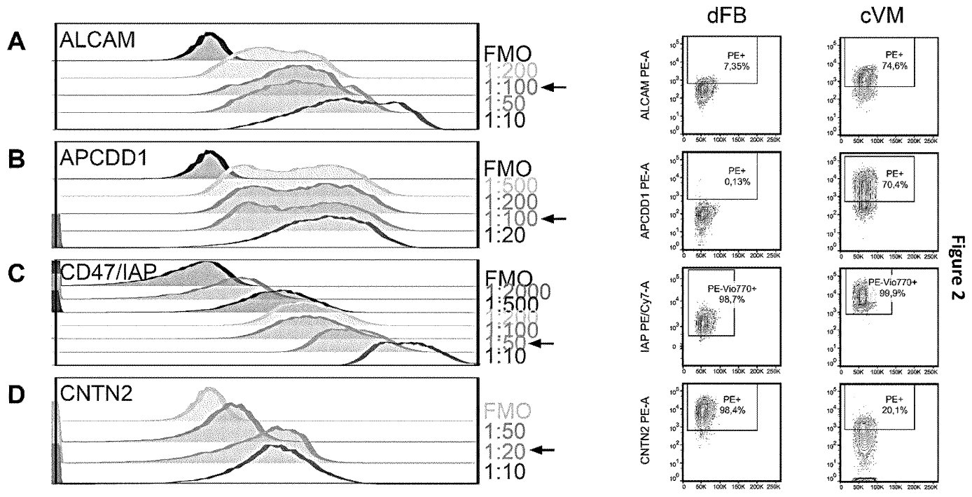

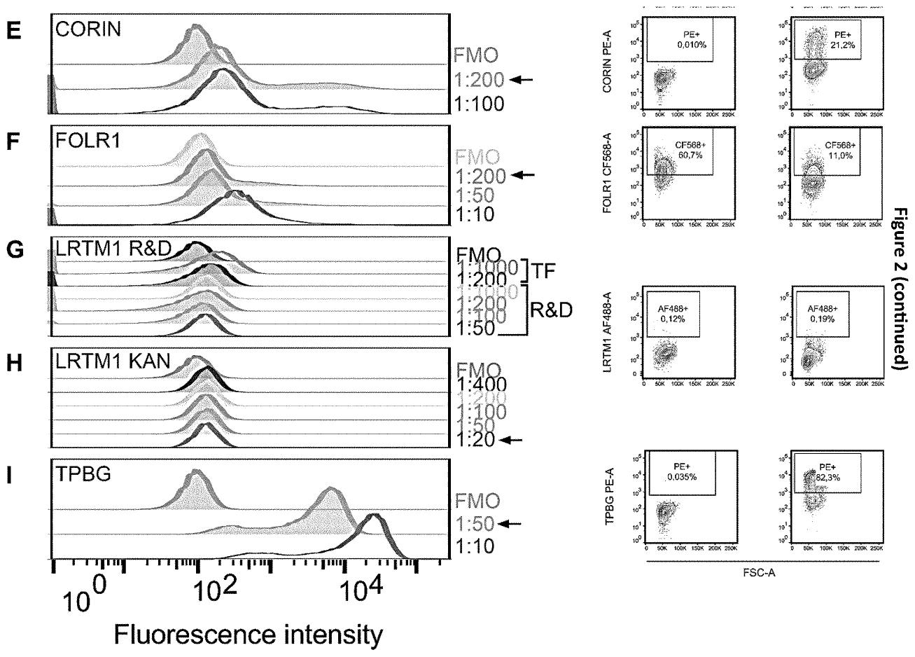

FIG. 1. Identification of candidate caudal ventral midbrain cell surface marker APCDD1.
A. Single-cell RNA sequencing of day 14 ventral MiSTR tissue plotted in UMAP dimensions. Expression of previously published caudal ventral midbrain cell surface marker genes ALCAM, LRTM1, CORIN, FOLR1, CD47/IAP, CNTN2, candidate marker genes TPBG and APCDD1, ventral midbrain genes FOXA2, LMX1A, OTX2 and EN1, and forebrain and hindbrain markers NKX2-1 and HOXB1, respectively.
B. Immunofluorescent labelling of EN1, APCDD1 and OTX2 in human foetal midbrain in gestational week (GW) 5.

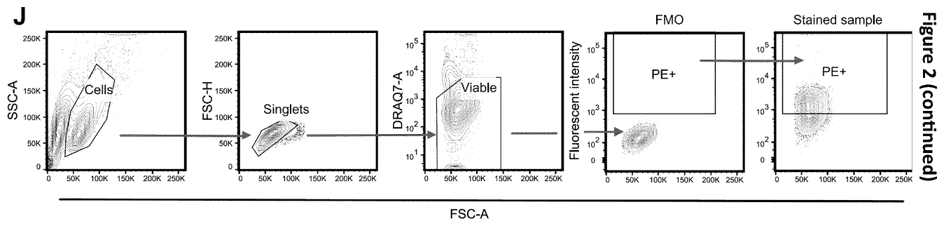

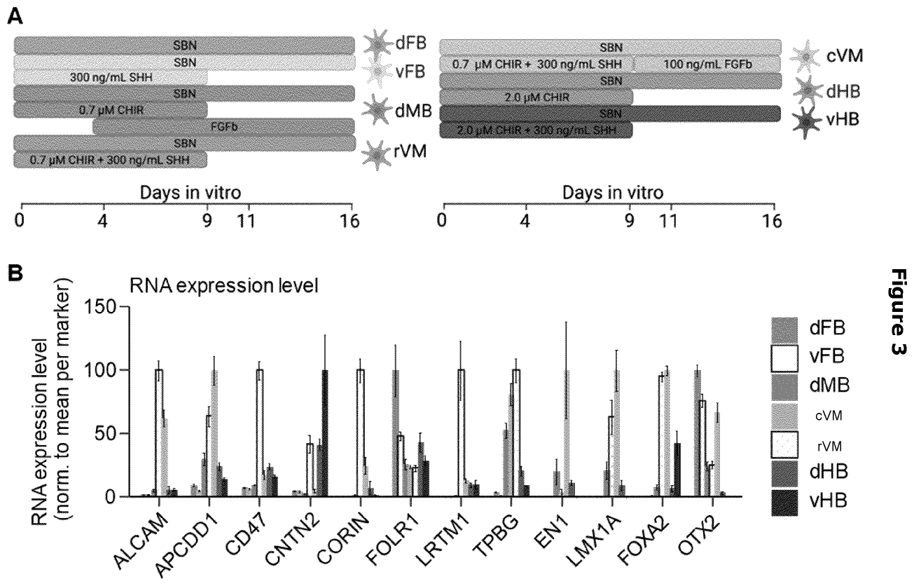

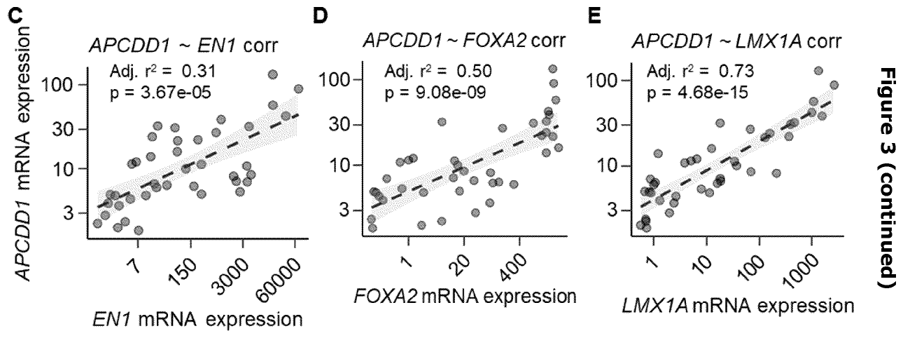

FIG. 2. Validation of flow cytometric regional specificity panel.
A-I. Titration of antibodies against caudal ventral midbrain (cVM) progenitor marker ALCAM (A), APCDD1 (b), CD47/IAP (C), CNTN2 (D), CORIN (e), FOLR1 (F), LRTM1 (G-H) and TPBG (I), and representative flow cytometry plots for dorsal forebrain (dFB) and cVM progenitors. The y-axis shows the modal-normalized frequency distribution. Suboptimal day 16 cVM progenitors were used for the titration. Selected concentrations are marked by arrows. Two anti-LRTM1 antibodies were tested. Firstly, an anti-LRTM1 from R&D Systems (G) for which two secondary antibodies were tested: anti-rat PE from Thermo Fisher (TF) and anti-rat PE from R&D. Secondly, an anti-LRTM1 from KAN Research Institute (KAN) (H).
J. Generic, fluorochrome-specific gating strategy used by using fluorescence minus one (FMO)-controls.

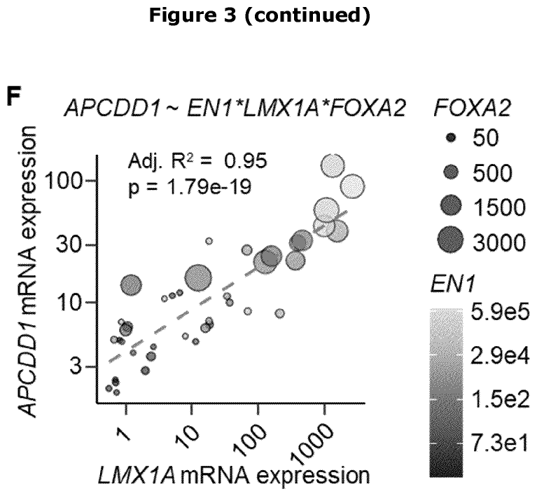

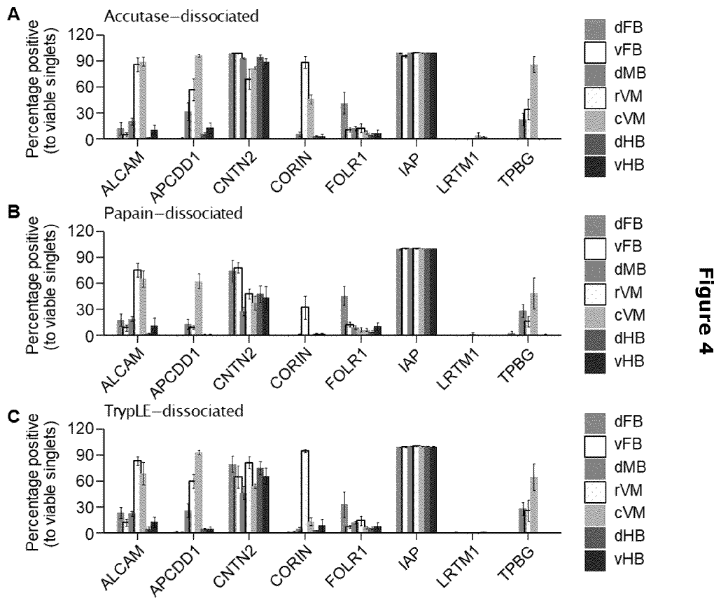

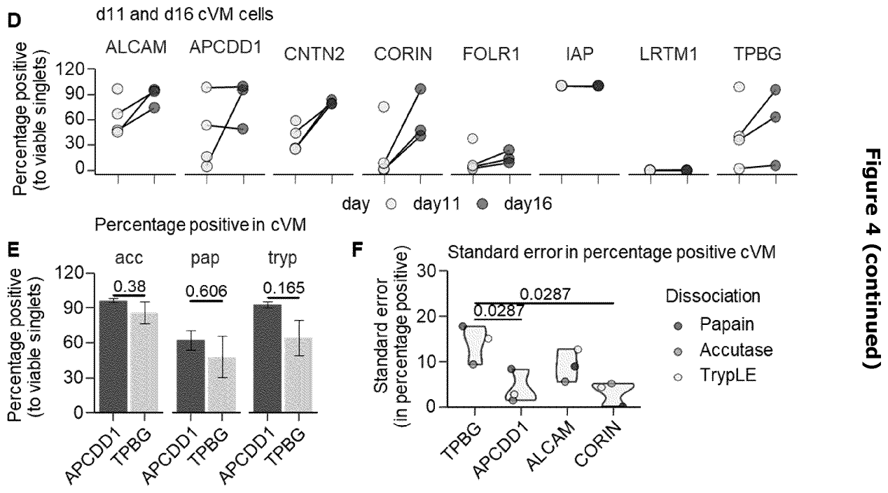

FIG. 3. RNA expression levels of APCDD1 and previously published caudal ventral midbrain cell surface markers.
A. Differentiation protocol schematic to produce dorsal forebrain (dFB), ventral forebrain (vFB), dorsal midbrain (dMB), rostral ventral midbrain (rVM), caudal ventral midbrain (cVM), dorsal hindbrain (dHB) and ventral hindbrain (vHB) day 16 neuronal progenitors.
B. Mean-normalized fold change mRNA expression levels, with a scaling value of 100, to undifferentiated RC17 hESCs for ventral midbrain dopaminergic progenitor cell surface marker genes in dFB, vFB, dMB, rVM, cVM, dHB and vHB day 16 neuronal progenitors (n≥4).
C-F. Linear regression of log 10-transformed fold change mRNA expression levels to undifferentiated RC17 hESCs in dFB, vFB, dMB, rVM, cVM, dHB and vHB.
C-E. Simple, linear regression models between APCDD1 and EN1 (C), APCDD1 and FOXA2 (D), and APCDD1 and LMXA1 (E).
F. Multiparametric, linear regression between APCDD1 and EN1, LMX1A, FOXA2 expression levels. Dots are sized in respect to the FOXA2, and colour-scaled by log 10-transformed-EN1 expression levels using the batlow palette.

FIG. 4. Benchmarking caudal ventral midbrain cell surface marker in flow cytometric regional panel.
A-C. Benchmarking cell surface markers indicative of ventral midbrain dopaminergic progenitors by flow cytometric analysis of dorsal forebrain (dFB), ventral forebrain (vFB), dorsal midbrain (dMB), rostral ventral midbrain (rVM), caudal ventral midbrain (cVM), dorsal hindbrain (dHB) and ventral hindbrain (vHB) day 16 neuronal progenitors.
A-C. Percentage of viable singlets positive for putative cell surface markers after dissociation with Accutase (A), Papain (B) and TrypLE (C) after 16-days differentiation in vitro. Bars are coloured by the region.
D. Percentage of viable singlets positive for putative surface markers after 11- and 16-days differentiation in vitro, and dissociation with Accutase from paired batches of cVM cells.
E. Percentage APCDD1⁺ and TPBG⁺ cells to viable singlets in day 16 cVM progenitors dissociated with Accutase, Papain and TrypLE, colored by the surface marker.
F. Standard error in percentage of positive viable singlets for Accutase- and TrypLE-dissociated day 16 cVM progenitor cells, grouped by the dissociation method and coloured by the region.
Error bars represent the standard error of the mean.

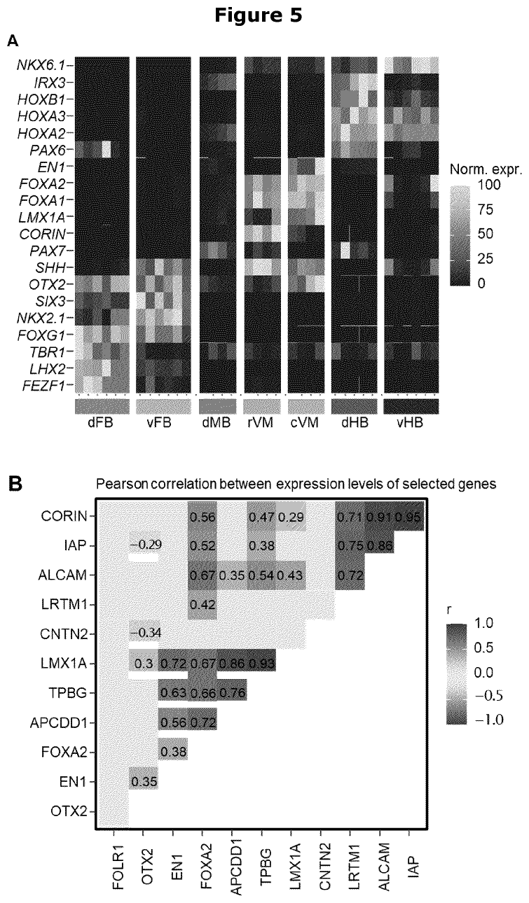

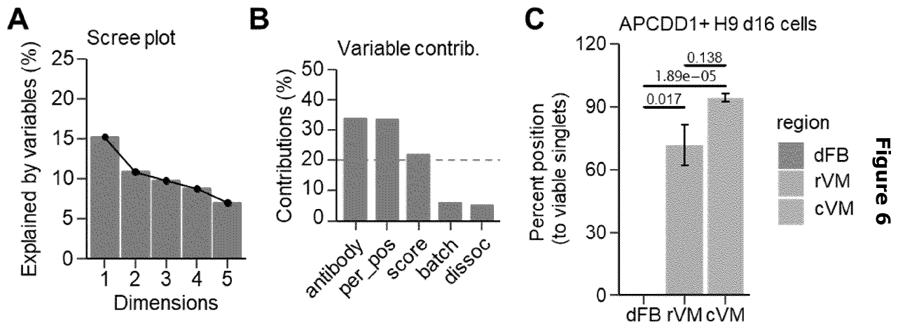

FIG. 5. mRNA levels in neuronal progenitor cells.
A-B. Fold change mRNA expression to undifferentiated RC17 hESCs for selected regional markers in dorsal forebrain (dFB), ventral forebrain (vFB), dorsal midbrain (dMB), rostral ventral midbrain (rVM), caudal ventral midbrain (cVM), dorsal hindbrain (dHB) and ventral hindbrain (vHB) day 16 neuronal progenitors (n≥4).
A. Heatmap of max-normalized RNA expression levels.
B. Pearson correlation between ventral midbrain transcription factors and surface markers. Non-significant regression models are signified by the lack of r-correlation coefficients.

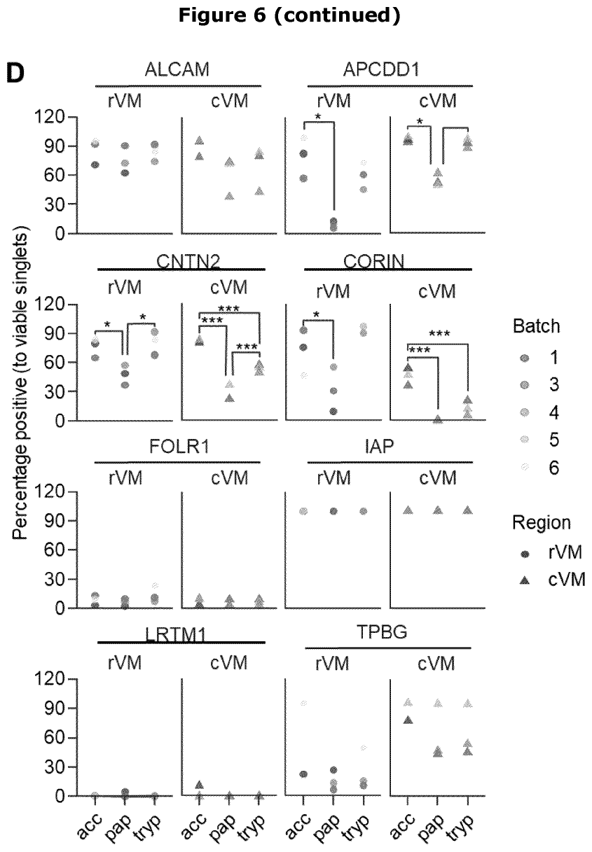

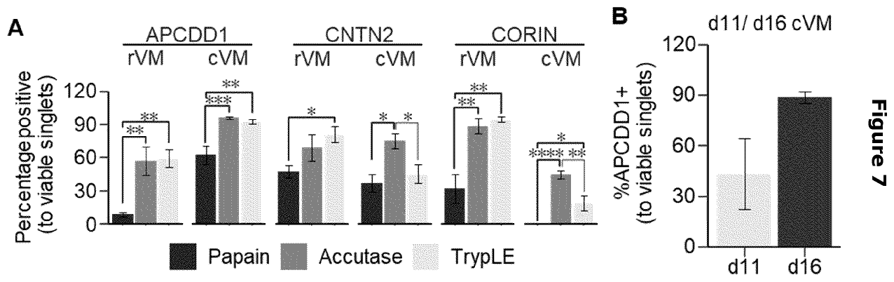

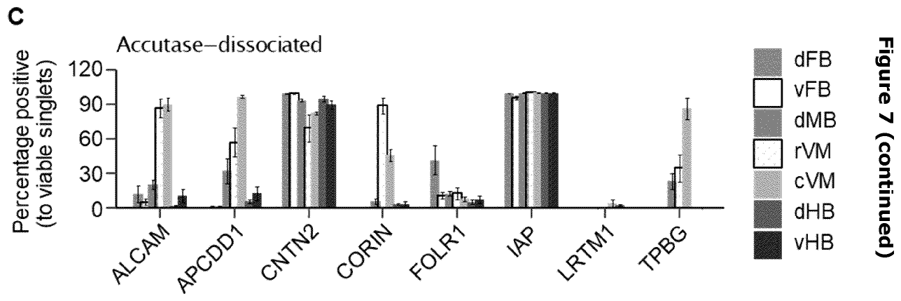

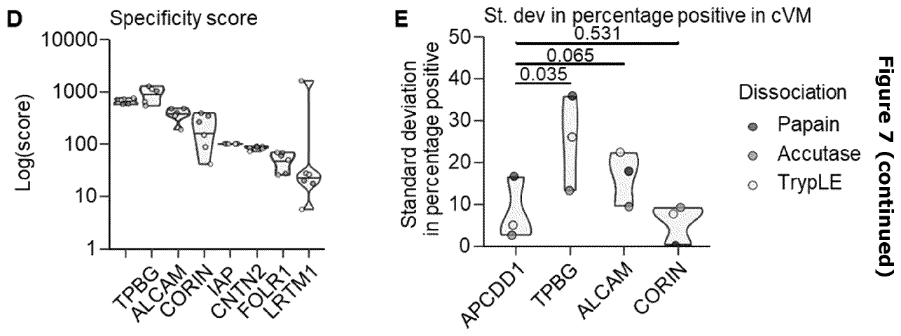

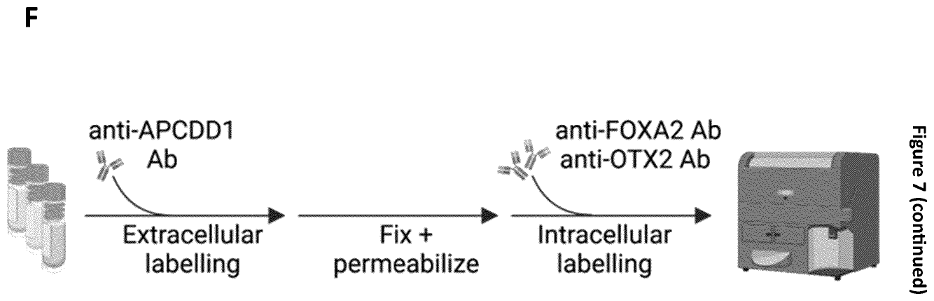

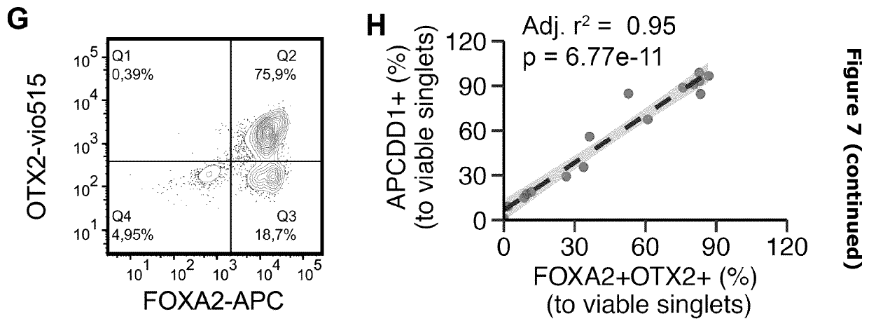

FIG. 6. The influence of the dissociation method for caudal ventral midbrain cell surface marker in flow cytometric regional panel and regional enrichment of H9 human embryonic stem cells.
A-B. Factor analysis for mixed data (FAMD) on flow cytometric regional panel.
A. Scree plot of the percentage of variance explained in each dimension in the percentage of viable singlets positive for putative surface markers.
B. Variable contribution in percent in the first dimension.
C. Percentage APCDD1⁺ viable singlets in H9-derived day 16 Accutase-dissociated dorsal forebrain (dFB), rostral ventral midbrain (rVM) and caudal ventral midbrain (cVM) cells.
D. Percentage positive day 16 rVM and cVM progenitor cells to viable singlets, grouped by dissociation method, faceted by cell surface markers and region. Points are shaped according to the region (rVM, dot; cVM, triangle) and coloured by the batch. The asterisk refers to statistically significant differences as determined by a Dunn's multiple comparison test (APCDD1) or pairwise t-tests (CNTN2 and CORIN) with Benjamini-Hochberg false-discovery rate detection. *, p≤0.05; **, p≤0.01; ***, p≤0.005.

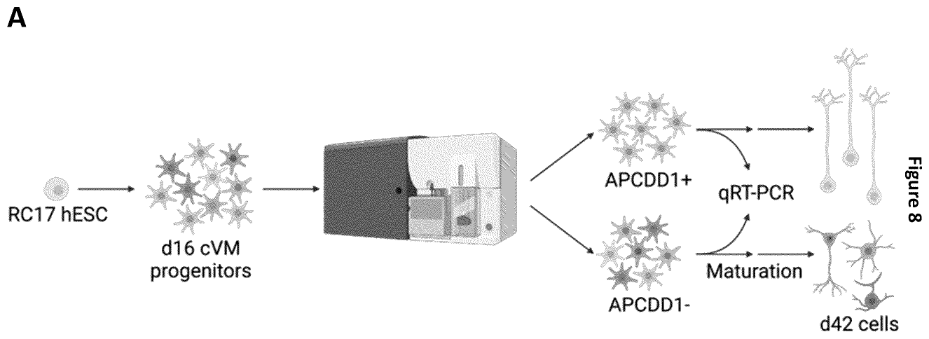

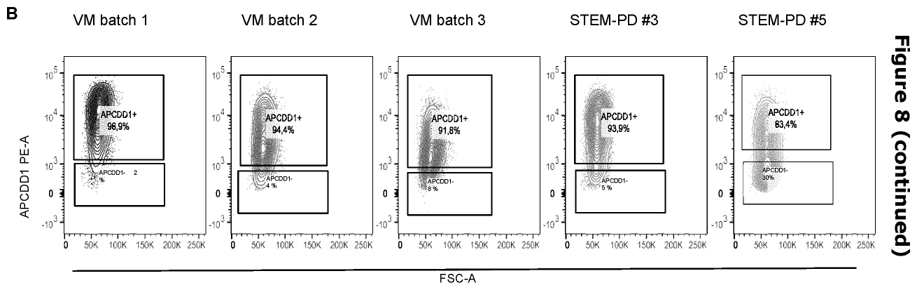

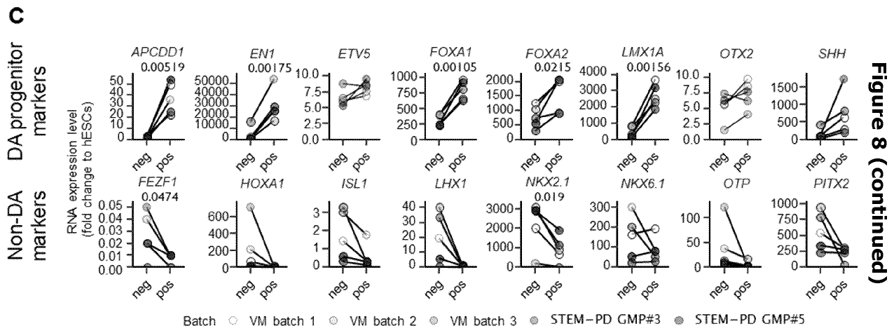

FIG. 7. Benchmarking of APCDD1 as caudal ventral midbrain cell surface marker in flow cytometric regional panel against published markers.
A-F. Benchmarking cell surface markers indicative of ventral midbrain dopaminergic progenitors by flow cytometric analysis of dorsal forebrain (dFB), ventral forebrain (vFB), dorsal midbrain (dMB), rostral ventral midbrain (rVM), caudal ventral midbrain (cVM), dorsal hindbrain (dHB) and ventral hindbrain (vHB) day 11 and 16 neuronal progenitor cells.
A. Percentage of positive day 16 rVM and cVM progenitors to viable singlets for surface markers that were affected by the dissociation method. The asterisk refers to statistical differences in the percentage of positive cells between the dissociation methods for either or both regions, determined by using region-nested, pairwise analysis of variance (ANOVA) tests.
B. Percentage APCDD1⁺ cells to viable singlets in day 11 and day 16 cVM Accutase-dissociation progenitors from paired batches (n=3).
C. Percentage of positive viable singlets in day 16 Accutase-dissociated cells, coloured by the region (n≥3).
D. Enrichment for cVM progenitors dissociated, as determined with a specificity index. Dots are coloured by the dissociation method (n=≥5).
E. Standard deviation (SD) in the percentage of positive singlets in rVM and cVM regions.

Dots are coloured by the dissociation method. APCDD1 had statistically significantly decreased SD compared to TPBG and ALCAM, determined by using paired, pairwise t-tests with Benjamini-Hochberg false-discovery rate correction.
F-H. Intracellular and extracellular flow cytometric analysis of day 16 cVM progenitors, using fully stained dFB progenitors as a biological negative control to set the FOXA2⁺/OTX2⁺ gates. Experiment schematic (F) FOXA2⁺/OTX2⁺ (Q2) (G), and linear regression model between the percentage of FOXA2⁺/OTX2⁺ and APCDD1⁺ to viable singlets (n=9) (H).
*, p≤0.05. Error bars represent the standard error of the mean.

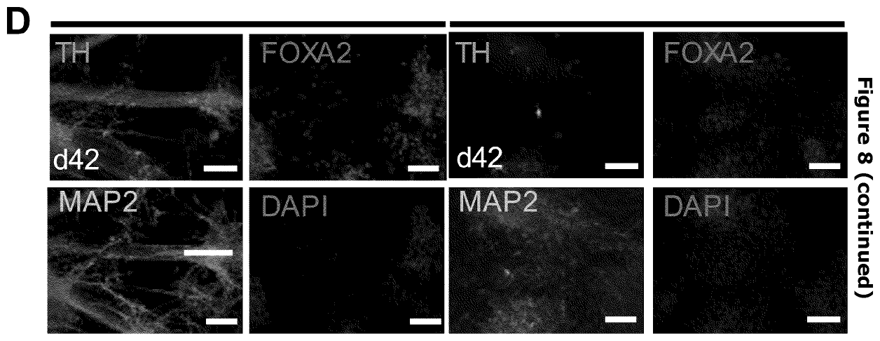

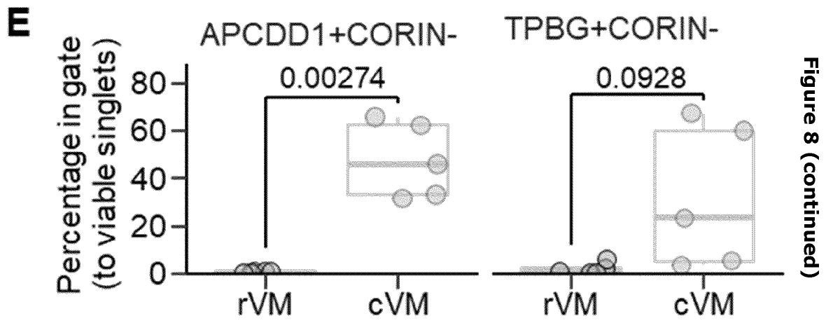

FIG. 8. Validation of APCDD1 association to a caudal ventral midbrain (VM) fate sorting pure cell differentiations and analysis of caudal VM to rostral VM enrichment.
A-D. Sorting pure caudal ventral midbrain (VM) batches for APCDD1.
A. Illustration of experimental design. Briefly, RC17-derived, high-purity cryopreserved day 16 caudal VM progenitor cells were sorted for APCDD1. The RNA was isolated directly post-sort, and the expression levels were analysed by using quantitative real-time PCR (qRT-PCR). Cells were also replated and fixed at day 42 for immunocytochemical (ICC) analysis.
B. Flow cytometry plot of the approximative % APCDD1^{+/-} cells in the VM batch 1-3 and clinical batch STEM-PD GMP #3 and #5.
C. mRNA fold change to undifferentiated human embryonic stem cells of VM and non-VM genes for APCDD1-sorted cells. Statistical significance was evaluated by using paired t-tests (n=5).
D. ICC of sorted, day 42-terminal differentiated cells using TH, FOXA2 and MAP2. Scale bar, 100 μm.
E. Flow cytometric analysis of rVM versus cVM regional specificity. The panel APCDD1⁺CORIN⁻ could best discriminate between rostral ventral midbrain (rVM) and caudal ventral midbrain (cVM).

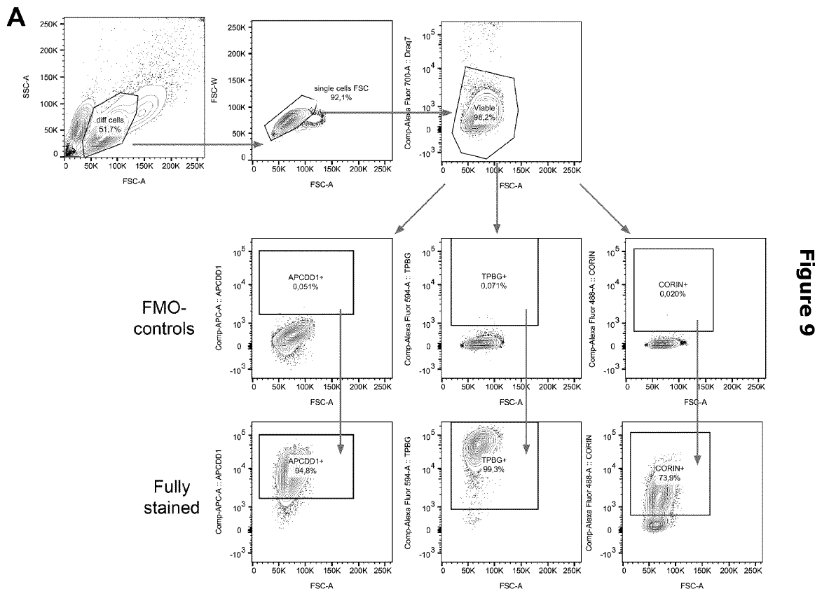

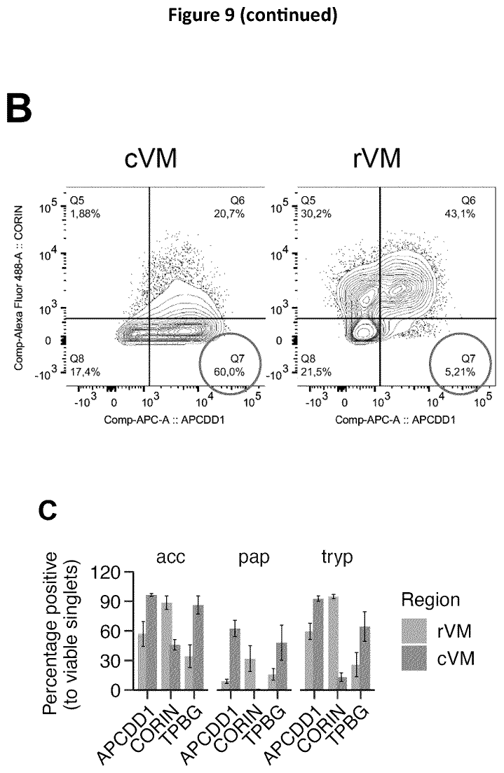

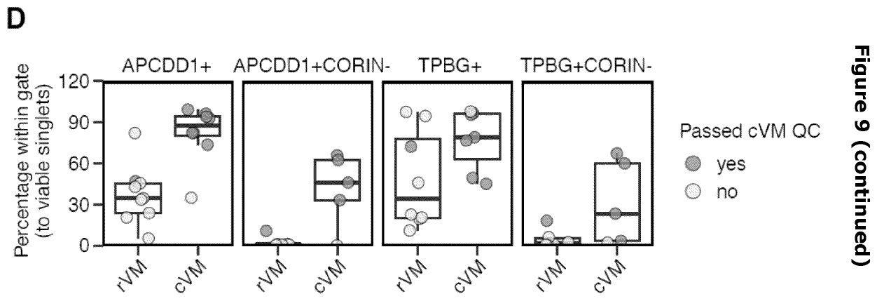

FIG. 9. Caudal ventral midbrain versus rostral central midbrain enrichment panel.
A. Generic, fluorochrome-specific gating strategy used by using fluorescence minus one (FMO)-controls.
B. Gating used for APCDD1⁺CORIN⁻ panel for caudal ventral midbrain (cVM, left) and rostral central midbrain (rVM, right) cells. See bottom right quadrant (circled).
C. Percentage positive cells to viable singlets in day 16 caudal ventral midbrain (cVM) and rostral central midbrain (rVM) progenitors dissociated with Accutase, Papain and TrypLE.
D. Percentage cells within the gate to viable singlets in day 16 cVM and rVM progenitors dissociated. Points are coloured according to whether the batch passed the cVM qRT-PCR quality control.

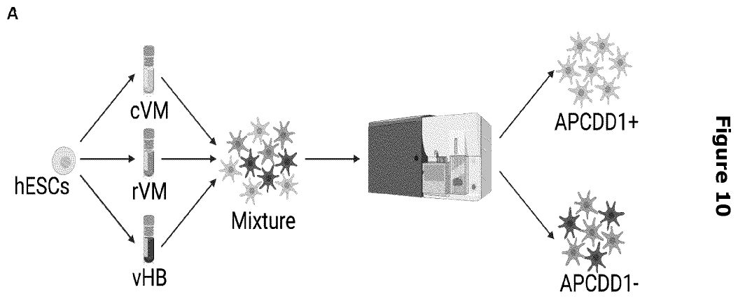

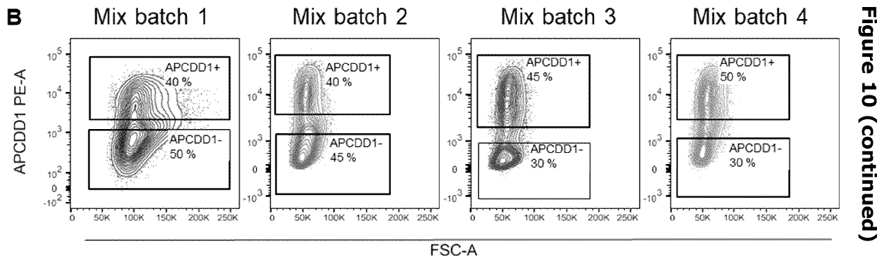

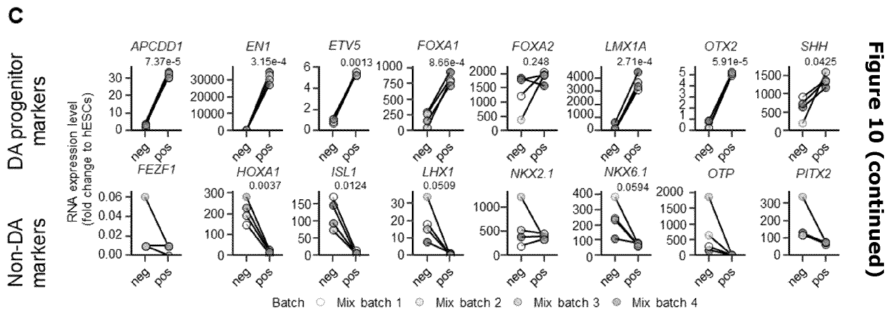

FIG. 10. Rescue of contaminated caudal ventral midbrain batches by sorting for APCDD1⁺ cells.
A-C. Sorting for APCDD1 in mixed batches of cells.
A. Illustration of experimental design. 4:3:3 mixtures of RC17 hESC-derived caudal ventral midbrain (cVM), rostral ventral midbrain (rVM) and ventral hindbrain (vHB) day 16 progenitor cells were sorted for APCDD1. The RNA was isolated directly post-sort, and the expression levels were analysed by using quantitative real-time PCR. Cells were transplanted to Parkinsonian nude rats.
B. Flow cytometry plots of approximative % APCDD1^{+/-} cells in the mixed batches.
C. mRNA fold change to undifferentiated human embryonic stem cells of VM and non-VM genes for APCDD1-sorted cells. Statistical significance was evaluated by using paired t-tests (n=4).

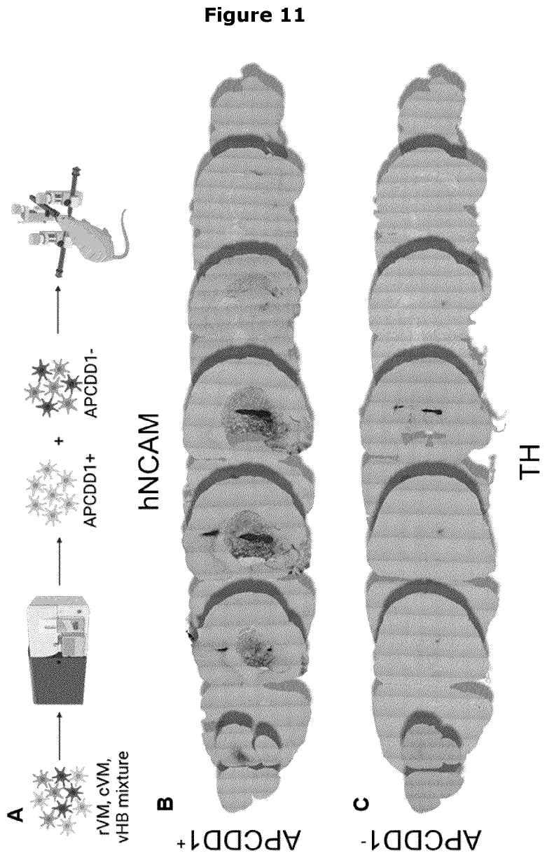

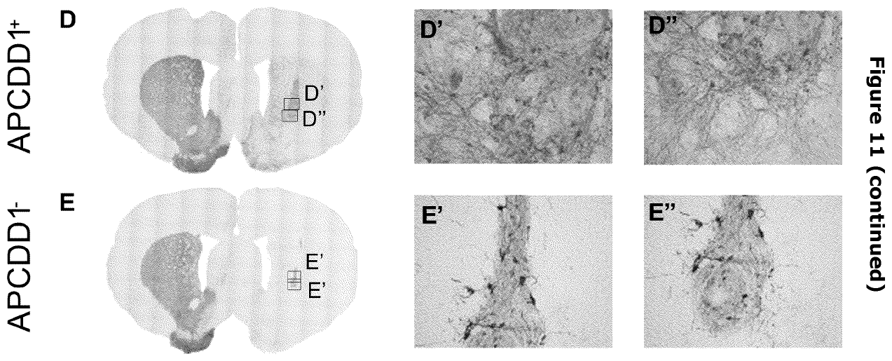

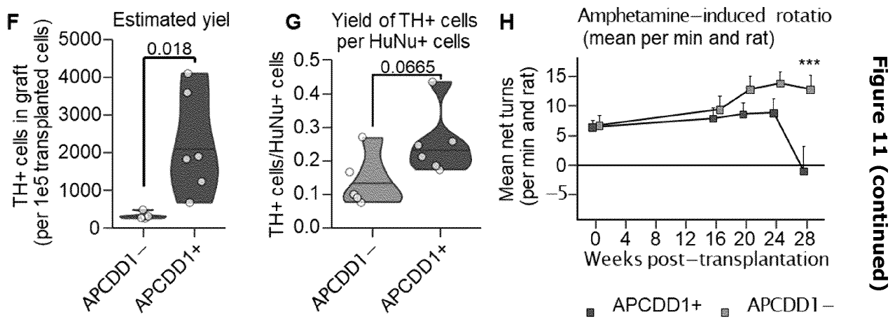

FIG. 11. Functional maturation of APCDD1⁺ cells to ventral midbrain dopaminergic neurons by cell sorting mixed batches of cells.
A-H. Sorting for APCDD1 in mixed batches of cells and transplanting to Parkinsonian nude rats.
A. Illustration of experimental design. 4:3:3 mixtures of RC17 hESC-derived caudal ventral midbrain (cVM), rostral ventral midbrain (rVM) and ventral hindbrain (vHB) day 16 progenitor cells were sorted for APCDD1. Cells were transplanted to the Parkinsonian nude rat striatum. Rats were sacrificed 28-weeks post-transplantation.
B-C. hNCAM di-amino benzidine (DAB) immunohistochemistry (IHC) in APCDD1⁺ (B) and APCDD1⁻ sorted cells (C).
D-E. TH DAB IHC in APCDD1⁺ (D) and APCDD1⁻ sorted cells (E).
F. The estimated total yield of TH⁺ cells per 1e5 cells transplanted.
G. The total number TH⁺/HuNu⁺ cells per animal.
H. Amphetamine-induced rotation test as 16-, 20-, 24 and 28-weeks post-transplantation.

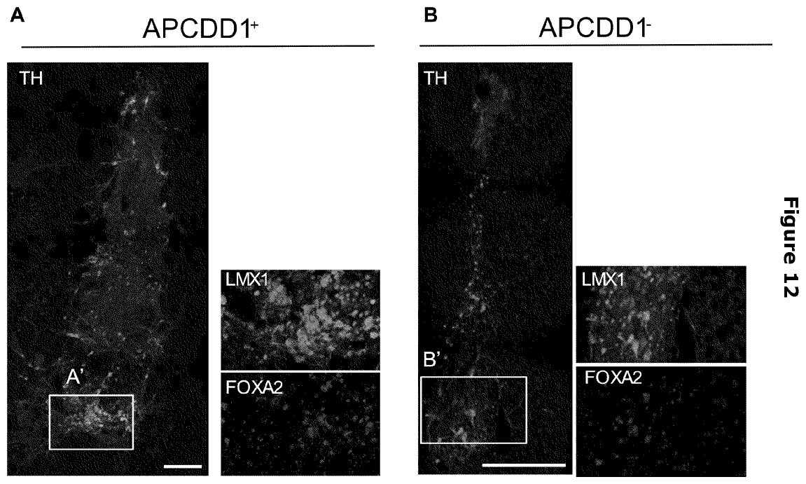

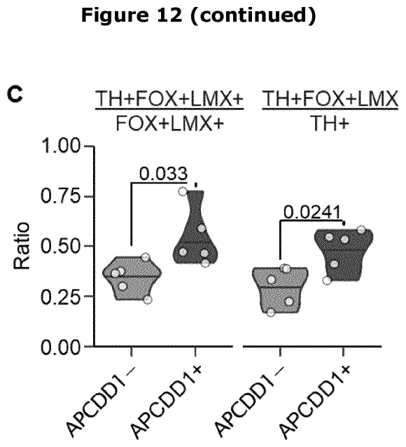

FIG. 12. Characterization of graft composition of APCDD1-derived transplants, sorted from mixed cells.
A-C. Characterisation of composition of APCDD1-sorted transplants derived from mixed batches of cells.
A-B. TH, LMX1A and FOXA2 immunofluorescence in APCDD1⁺ (A) and APCDD1⁻ (B) sorted cells.
C. Quantification of TH, LMX1A and FOXA2, single-, double, and triple-positive cells in APCDD1-derived grafts. Statistical significance was evaluated by using t-tests (n=5).

### EXAMPLES

The inventors identified adenomatosis polyposis coli down-regulated 1 (APCDD1) as a novel cell-surface candidate biomarker predictive of functional maturation of cVM progenitors to ventral midbrain (VM) dopaminergic (DA) neurons.

### BACKGROUND AND SUMMARY

Parkinson's disease (PD) is common neurodegenerative movement disorder. Levodopa is the most common treatment strategy for ameliorating the motor symptoms of this disease, but is associated with serious complications and the efficiency fluctuates and progressively diminishes (Connolly and Lang 2014). Since the motor symptoms of PD can be attributed to a relatively selective and focal loss of dopaminergic (DA) neurons within the ventral midbrain (VM), cell replacement is a promising alternative treatment strategy to levodopa.

The feasibility of this approach has been demonstrated by using fetal VM-derived cells (Barker et al. 2019; Björklund et al. 1980; Bolam et al. 1987; Dunnett et al. 1983; Freund et al. 1985; Strecker et al. 1987). Due to tissues-related issues however, the focus has recently shifted towards using human pluripotent stem cells (hPSCs) as a near inexhaustible source of VM DA cells (Kefalopoulou et al. 2014; Kurowska et al. 2011; J. Y. Li et al. 2008; W. Li et al. 2016). The cells are transplanted as progenitors that mature in the host brain to replace the functions of endogenously lost cells.

Human pluripotent stem cells are a promising source of caudal ventral midbrain (cVM) progenitor cells that are used in cell replacement therapy for Parkinson's disease. However, some heterogeneity in cell composition is inevitable in the cell differentiation process. Cell surface markers predictive of functional maturation of cVM progenitors to ventral midbrain (VM) dopaminergic (DA) neurons could be used as a convenient quality control and to enrich for the target cell type.

A pure population of cells must be transplanted for safe, efficacious, and reproducible outcome. Genes indicative of VM DA progenitors, such as the transcription factors LMX1A, FOXA2 and OTX2, are commonly used surrogate markers for functional maturation (Arenas, Denham, and Villaescusa 2015). These markers are incorporated in intracellularly based flow cytometric quality control (QC) panels that are assessing the purity of the differentiated cells. However, only caudal VM (cVM)-derived LMX1A/FOXA2/OTX2 triple-positive progenitors give rise to VM DA neurons whereas triple-positive cells of the rostrally-adjacent subthalamic nucleus (STN), referred to as the rostral VM (rVM) from this point, produces glutamatergic neurons (Nolbrant et al. 2017). Current flow cytometry-based QC assays using LMX1A/FOXA2/OTX2 are unable to distinguish between cVM and rVM fates. Indeed, FOXA2, OTX2 and LMX1A expression levels fail to predict graft outcome upon transplantation (Kirkeby et al. 2017). Although EN1 specifically marks a VM DA fate, there is not a sufficiently specific EN1 antibody available for flow cytometry. Further, intracellular flow cytometry is associated with increased sample processing time and considerable unspecific background staining compared to using cell surface markers, complicating gating in a good manufacturing practice (GMP)-compliant setting. Indeed, only gating to fluorescence minus one (FMO)-controls are allowed within a GMP setting, which fail to correspond to the de facto background observed for intracellular flow cytometry. Extracellular-based flow cytometry would be a more convenient QC of cell differentiations than intracellular flow cytometry as it would circumvent to caveat listed above. Additionally, due to the lack of fixation and permeabilization, the cells could also be sorted for a VM DA fate while eliminating undesired cells, such as serotonergic and stem cells. This would enable direct correlation between the cell product and graft outcome, which is currently not possible. Lastly, surface markers could be used to enrich for the target population in induced PSC (iPSC)-derived differentiations immediately before transplantation.

There are several published, putative surface markers predictive of VM DA neurons (Bye et al. 2015; Doi et al. 2014; Fathi et al. 2018; Gennet et al. 2016; Kikuchi et al. 2017; Lehnen et al. 2017; Paik et al. 2018; Samata et al. 2016; Sundberg et al. 2013; Yoo et al. 2021), some of which ameliorate behavioural deficits in Parkinsonian animal models (Doi et al. 2014; Kikuchi et al. 2017; Lehnen et al. 2017; Sundberg et al. 2013; Yoo et al. 2021). Apart from the surface marker CORIN, few of these have been extensively characterized, and the regional specificity is underexplored. See Table 3 for summary of surface markers.

Whereas several of these studies have focused on the enrichment of LMX1A⁺ and FOXA2⁺ cells from a mixed starting progenitor population, few have focused on benchmarking the specificity of the markers against a range of other regional neural progenitor cells to scrutinize the specificity of the markers against potentially contaminating populations of other neural fates. In addition, there has been little focus on assessing the specificity of the published markers for the true DA progenitor cells of the cVM, which are LMX1A⁺FOXA2⁺EN1⁺ versus the neighbouring non-DA rVM progenitor, which are LMX1A⁺FOXA2⁺EN1⁻ (Kee et al. 2017; Kirkeby et al. 2017). The inventors therefore identified a need for comparison of cell surface markers vis-à-vis to identify markers truly enriched for a bona fide VM DA fate to aid the development of accurate and efficient QC assays of cell products that will be used in phase-I clinical trials exploring cell replacement therapy as a PD treatment.

The inventors provide herein a novel candidate marker predictive of VM DA neurons-adenomatosis polyposis coli down-regulated 1 (APCDD1), which functions as a Wnt/β-catenin antagonist (He and Tang 2020; Kagermeier-Schenk et al. 2011; Shimomura et al. 2010; Zimmerli et al. 2020). By comparing the regional specificity of previously published, putative surface makers of VM DA progenitors to APCDD1 and TPBG by using quantitative real-time PCR (qRT-PCR), flow cytometry and modelling of the regional enrichment, the inventors have shown that the newly identified surface markers were the most enriched for a VM DA fate. APCDD1 correlates to EN1 on a transcriptomic level as well as the percentage of APCDD1⁺ cells to FOXA2⁺/OTX2⁺ cells. Animals transplanted with APCDD1⁺ cells, sorted from a mixed population of cells, had complete behavioural recovery in a rat model of PD, whereas animals transplanted with APCDD1⁻ cells had no improvement. Grafts derived from APCDD1⁺ cells had a higher yield of bona fide VM DA neurons compared to grafts from APCDD1⁻ cells.

APCDD1 is the most predictive surface marker of a VM DA neuronal fate, and thereby represents an attractive candidate for flow cytometry-based QC assays for cell replacement therapies for PD.

### Example 1

### Materials and Methods

hESCs Differentiation in MiSTR

Roslin Cells 17 (RC17, hPSC reg #RCe021-A) hESCs were cultured and differentiated in the custom-designed microfluidics device as detailed in Rifes et al. 2020 (Rifes et al. 2020). Briefly, by using a continuous flow of neural patterning medium (NPM), consisting of 1:1 mix of DMEM/F12 (Invitrogen) and NeuroMedium (Miltenyi Biotec), N2 supplement (1:200; Invitrogen), NeuroBrew-21 without vitamin A (1:100), supplemented with 10 μM SB431542 (Miltenyi Biotec) and 100 ng/mL rh-Noggin (Miltenyi Biotec). One syringe contained medium with added GSK3i (100% side, CHIR99021, Miltenyi Biotec), the other without GSK3i (0%). For ventralization, we added a 200 ng/mL SHH-C24II (Miltenyi Biotec) and 0.5 μM purmorphamine (Miltenyi Biotec) to the medium of both inlets.

hESC Culturing and Differentiation

RC17 hESCs were differentiated towards progenitors of dorsal forebrain (dFB), ventral forebrain (vFB), dorsal midbrain (dMB), rVM, cVM, dorsal hindbrain (dHB), and ventral hindbrain (vHB) fates. For all conditions, media composition, coating, seeding densities and replating steps were followed as described in Nolbrant et al., 2017 until d16, as previously described (Kirkeby et al. 2017; Nolbrant et al. 2017). All conditions received dual SMAD inhibition (SB431542 10 μM and Noggin 100 ng/ml) from day 0-9 of differentiation. Patterning into each of the different regions was obtained by differential addition of patterning factors CHIR99021 (referred to as CHIR), SHH-C24II (referred to as SHH) and FGF8b, all from Miltenyi Biotec, as follows: dFB (no additional factors added), vFB (SHH 300 ng/ml day 0-9), dMB (CHIR 0.7 uM day 0-9+FGF8b 100 ng/ml day 4-16), rVM (CHIR 0.7 μM day 0-9+SHH 300 ng/ml day 0-9), cVM (CHIR 0.7 μM day 0-9+SHH 300 ng/ml day 0-9+FGF8b 100 ng/ml day 9-16), dHB (CHIR 2 μM day 0-9) and vHB (CHIR 2 μM day 0-9+SHH 300 ng/ml day 0-9). H9 hESCs (WA09, WiCell Research Institute, Inc.) were differentiated to dFB, rVM and cVM progenitors similarly as for RC17 with some alterations. The day 0 seeding density was 15,000 cells/cm². The following patterning factors were added: dFB (N2+10 μM SB+100 ng/mL Noggin day 0-9, N2 day 9-11, B27+0.2 mM AA+20 ng/ml BDNF day 11-16), rVM (N2+10 μM SB+100 ng/ml Noggin+500 ng/ml SHH+0.7 uM CHIR day 0-9, N2 day 9-11, B27+0.2 mM AA+20 ng/ml BDNF day 11-16), cVM (N2+10 μM SB+100 ng/ml Noggin+500 ng/ml SHH+0.7 μM CHIR day 0-9, N2+100 ng/ml FGF8b day 9-11, B27+0.2 mM AA+20 ng/ml BDNF+100 ng/ml FGF8b day 11-16). The cells were harvested on day 11 with Accutase (ThermoFisher) or at day 16 with either Accutase, TrypLE (ThermoFisher) or Neural Tissue Dissociation kit (P) from Miltenyi (referred to as Papain)—all using 10 minutes of enzymatic incubation. After dissociation on day 16, cells were cryopreserved in CryoStor CS10 (Sigma) for later use in flow cytometry. For terminal maturation, cells were kept on laminin-521-coated plates from day 16 and onwards and cultured in terminal differentiation medium as described in Nolbrant et al. 2017.

Library Preparation, Sequencing, Processing, Alignment and Analysis of scRNA-Seq Data

scRNA-seq of the MiSTR tissue, was performed as detailed in Rifes et al. 2020 (Rifes et al. 2020). In short, cells were loaded per lane onto the 10× Chromium (10× Genomics), and cDNA libraries were generated according to manufacturer's instructions. Cells from five regions were mixed in equal ratios and were then loaded on 10× lane using 10×V3 chemistry Kit. cDNA sequencing was performed on a NextSeq 500 (Illumina) according to the manufacturer's instructions. Post-processing, alignment, and analysis of the scRNA-seq data from the MiSTR tissue was performed. Uniform manifold approximation and projectionx (UMAP)s were visualized by using RunUMAP( ) function of Seurat based on pre-computed principal components (PCAs).

### Flow Cytometry

For the regional specificity panel, the same concentration and manufacturer of published antibodies were used to the largest extent possible and as bright fluorophores as possible. The LRTM1 antibody (KAN Research Institute) was acquired thanks to a material transfer agreement with the supplier. Antibodies were reconstituted according to the manufacturers' instructions. For the regional specificity panel, cryopreserved cells patterned toward neuronal fates were thawed and resuspended in Buffer A (0.5% (vol/vol) KnockOut™ Serum Replacement (KOSR), Dulbecco's phosphate-buffered saline (DPBS) (—Ca²⁺/—Mg²⁺)). The cells were centrifuged and were then resuspended in Buffer B (2.5% (vol/vol) KOSR, DPBS (—Ca²⁺/—Mg²⁺)) to a final concentration of 1.0 million cells/mL. The cells were incubated with primary antibodies (CORIN, 1:200; LRTM1, 1:20/1:200; CNTN2, 1:20; IAP-PE/Vio770, 1:50; ALCAM-PE, 1:100; FOLR1, 1:200, conjugated with Mix-n-Stain™ CF® 568 Dye Antibody Labelling Kit according to the manufacturer's instructions; TPBG-PE, 1:50; APCDD1-PE, 1:100) indirectly on ice for 30 min, covered, on a slowly rotating platform. Cells were thereafter washed trice in Buffer A following centrifugation for 5 min at 500×g and 4° C. Goat anti-rabbit PE (1:200), mouse anti-rat PE (1:200) or donkey anti-rat AF488 (1:500) secondary antibodies were added to cells labelled with CNTN2, CORIN and LRTM1, respectively. The cells were thereafter washed as above. During the final resuspension, the cells were resuspended in the Buffer A+DRAQ7 (1:1,000) and were analysed on a BD FACSAriaIII™.

The rVM versus cVM panel was performed as described for the regional specificity panel above, using rVM and cVM progenitor cells from the same batch. Cells were incubated with APCDD1-APC (1:100), TPBG (1:200), CORIN (1:200), ZIP8 (1:500) and CD83-PE/Cy7 (1:100) antibodies. Cells stained with TPBG, CORIN and ZIP8 were then incubated with anti-mouse AF594, anti-rat AF488, anti-rabbit PE, respectively, as instructed above. Compensation controls were prepared by using Ultracomp eBeads™ Plus (BD Biosciences), according to the manufacturer's instructions. Cells were resuspended and washed in Buffer C (0.5% (vol/vol) human serum albumin (HSA), Hank's balanced salt solution (HBSS) (—Ca²⁺/—Mg²⁺)) and were stained in Buffer D (2.5% (vol/vol) HSA, HBSS (—Ca²⁺/—Mg²⁺)). During the final resuspension, the cells were resuspended in the Buffer C+DRAQ7 (1:1,000) and were analysed on a BD FACSAriaIII™.

For intracellular flow cytometry protocol, the cells were firstly prepared, stained with an APCDD1-PE antibody, and washed, as instructed for the rVM versus cVM panel above. Then, cells were diluted to 1.0 million cells/mL in Buffer E (1% (vol/vol) N-2™ supplement, CTS™ Neurobasal™ Medium). LIVE/DEAD™ Fixable Violet dye (Thermo Fisher) or dimethyl sulfoxide (DMSO) was added, and the samples were incubated for 15 min at room-temperature. After a wash in Buffer F (1% (vol/vol) bovine serum albumin (BSA), DPBS (—Ca²⁺/—Mg²⁺)), cells were fixed and permeabilized by the addition of 1×Perm/Wash buffer (BD Biosciences), prepared according to the manufacturer's instructions. After two sequential washes in 1×Perm/wash buffer (BD Biosciences), prepared as instructed, cells were stored at 4° C. over night, protected from light. Cells were then incubated in FOXA2-APC (1:80) and/or OTX2-Vio515 (1:320) antibodies for 30 min at 4° C. Cells were washed once in 1×Perm/wash buffer, and twice in Buffer F. Anti-REA beads (Miltenyi Biotec) were used for FOXA2-APC and OTX2-vio515, ArC-reactive beads (Miltenyi Biotec) for Live/Dead Fixable Violet Dye, and Ultracomp eBeads™ Plus (BD Biosciences) for APCDD1-PE single-stained controls, respectively, all prepared as recommended. Cells were interrogated on a BD LSRFortessa™, using fully stained dorsal forebrain or ventral hindbrain progenitor cells as a biological negative control to set the FOXA2⁺/OTX2⁺ gates.

During FACS, the cells were prepared as for the regional specificity panel. However, the cells were resuspended and washed in Buffer C and stained in Buffer D, respectively. During the final resuspension, when sorting cells on a BD FACSAria3™ or MACSQuant® Tyto®, the cells were resuspended in the Buffer C+DRAQ7 (1:1,000), and, when using a BD FACSMelody™, Buffer C+100 nM DAPI. Sorting for transplantation to SD rats was performed on BD FACSAria3™ from d16 cVM progenitor cells. Sorting for transplantation to nude rats was performed on BD FACSMelody™ from a mixture of cVM, rVM and vHB d16 progenitors, which were combined with a cell numeric ratio of 4:3:3. The cells were stained with an APCDD1-PE antibody to yield approximately 50% APCDD1⁺ cells to viable singlets, based upon previous flow cytometric analysis of the cells. The upper 40% APCDD1⁺ and lower 40% APCDD1⁻ fractions of viable cells were sorted. Cells to be transplanted were prepared as previously described in Nolbrant et al. 2017, and were transplanted immediately post-sort.

Gates were set based upon FMO controls, which were collected for each cell population during each analysis, and the compensation was determined by using single-stained controls. For each sample, ≥10,000 singlets were analysed. The following, generic gating strategy was used: (i) FSC-A vs. SSC-A, (ii) FSC-A vs. FSC-W, and (iii) FSC-A vs. live/dead-A. The gating for the regional specificity panel was performed in the FACSDiva™ software, and the statistics were thereafter exported. Post-processing, QC, and analysis of the exported .fcs files was done by using the software program FlowJo (version 10.7.2). The results are presented as percentage cells positive for the marker to parent (usual viable singlets). A list of reagents and dilutions used for flow cytometry can be found in Table 1.

TABLE 1
List of reagents and the dilutions. FC, flow cytometry; Di-amino benzidine,
DAB; IHC, immunohistochemistry; ICC, immunocytochemistry.

| Reagent | Dilution | Manufacturer | Cat. # | Application |
|---|---|---|---|---|
| LRTM1 | 1:200 | R&D Systems | MAB10046 | FC |
| LRTM1 | 1:20 | KAN Research Institute | — | FC |
| CORIN | 1:200 | R&D Systems | MAB2209 | FC |
| IAP-PE/Vio770 | 1:50 | Miltenyi Biotec | 130-101-358 | FC |
| CNTN2 | 1:20 | Abcam | EPR5106 | FC |
| ALCAM-PE | 1:100 | BD Biosciences | 560903 | FC |
| FOLR1 | 1:200 | R&D Systems | AF5646-SP | FC |
| TPBG-PE | 1:50 | R&D Systems | FAB49751P | FC |
| TPBG | 1:200 | R&D Systems | MAB49751 | FC |
| APCDD1-PE | 1:100 | Biolegend | 367303 | FC |
| APCDD1-APC | 1:100 | Miltenyi Biotec | 130-114-668 | FC |
| FOXA2-APC | 1:80 | Miltenyi Biotec | 130-124-043 | FC |
| OTX2-Vio515 | 1:320 | Miltenyi Biotec | 130-121-202 | FC |
| CD83-PE/Cy7 | 1:100 | BD Biosciences | 561132 | FC |
| ZIP8 | 1:500 | Proteintech | 20459-1-AP | FC |
| hNCAM | 1:500 | Santa Cruz | Sc-106 | IHC |
| | | Biotechnology | | |
| hCOL1A1 | 1:1,000 | R&D Systems | AF6220 | IHC/ICC |
| TH | 1:2,000 | Merck Millipore | AB152 | IHC |
| TH | 1:5,000 | Immunostar | 22941 | ICC |
| TH | 1:1000 | Merck Millipore | AB1542 | IHC/ICC |
| HuNu | 1:200 | Merck Millipore | MAB1281 | IHC |
| FOXA2 | 1:400 | R&D Systems | AF2400 | ICC |
| FOXA2 | 1:500 | Santa Cruz | sc-101060 | ICC |
| | | Biotechnology | | |
| LMX1A | 1:1,000 | Merck Millipore | AB10533 | ICC |
| OTX2 | 1:2,000 | R&D Systems | AF1979 | ICC |
| MAP2 | 1:500 | Merck Millipore | M1406 | ICC |
| MAP2 | 1:500 | Abcam | ab5392 | ICC |
| EN1 | 1:100 | Sigma Aldrich | HPA073141 | ICC |
| SOX1 | 1:100 | Cell Signaling | 4194 | ICC |
| | | Technology | | |
| ISL1/2 | 1:50 | DSHB | 39.4D5 | ICC |
| GFAP (STEM123/ | 1:500 | Takara Bio | Y40420 | ICC |
| SC123) | | | | |
| Donkey anti-mouse | 1:500 | Jackson | 715-585-150 | FC/ICC |
| AF594 | | ImmunoResearch | | |
| Donkey anti-rat | 1:500 | Jackson | 712-545-150 | FC |
| AF488 | | ImmunoResearch | | |
| Goat anti-rabbit PE | 1:10 | R&D Systems | F0110 | FC |
| Donkey anti-rabbit | 1:200 | Invitrogen | 12-4739-81 | FC |
| PE | | | | |
| Mouse anti-rat | 1:200 | Thermo Fisher | 12-4817-82 | FC |
| IgG2a PE | | | | |
| Mix-n-Stain ™ | — | Biotium | 92447 | FC |
| CF ® 568 Dye | | | | |
| Antibody Labelling | | | | |
| Kit | | | | |
| DRAQ7 | 1:1,000 | BD Biosciences | 51-9011172 | FC |
| DAPI | 1:1,000 | Sigma Aldrich | D9542 | FC/ICC |
| LIVE/DEAD ™ | 1:1,000 | Thermo Fisher | L34955 | FC |
| Fixable Violet dye | | | | |
| Ultracomp eBeads ™ | — | Thermo Fisher | 01-3333-42 | FC |
| Plus | | | | |
| Anti-REA | — | Miltenyi Biotec | 130-104-693 | FC |
| Compensation Beads | | | | |
| ArC Amine Reactive | — | Thermo Fisher | A10346 | FC |
| Compensation Kit | | | | |
| Gill II hematoxylin | — | Sigma Aldrich | GHS232-1L | IHC |

qRT-PCR

The RNA was isolated from d11 and d16 neuronal progenitor cells by using the RNeasy® Plus Micro Kit (Qiagen). The cDNA synthesized by using the Maxima First Strand cDNA Synthesis Kit for RT-qPCR (Thermo Fisher). qRT-PCR was performed by using Sybr Green Mastermix (Roche Life Sciences) and primers by using the automated pipetting instrument Bravo (Agilent) or a liquid handler I-DOT (Dispendix), and samples were analyzed on a LightCycler 480 instrument (Roche Life Sciences) by using a two-step protocol with a 60° C. annealing/elongation step. Samples were run in technical duplicates, and the averaged Ct values were used for calculations. Data are represented using the DDCt method. Fold changes was based upon the mean fold change to two housekeeping genes (ACTB and GAPDH) in undifferentiated RC17 hESCs. The data in the heatmap and the regional panel mRNA expression graph were normalized to the maximum fold change for each gene, with a scaling factor of 100. The primers used are summarized in Table 2. Only batches of cells that passed the stringent QC criteria, based upon the qRT-PCR data, were included in the study.

TABLE 2
List of primers included in qRT-PCR panel.

| Gene | Full gene name | Primer sequence (fwd/rev) |
|---|---|---|
| CORIN | Corin, serine peptidase | CATATCTCCATCGCCTCAGTTG/ |
| | | GGCAGGAGTCCATGACTGT |
| EN1 | Engrailed homeobox 1 | CGTGGCTTACTCCCCATTTA/ |
| | | TCTCGCTGTCTCTCCCTCTC |
| EN2 | Engrailed homeobox 2 | CCTCCTGCTCCTCCTTTCTT/ |
| | | GACGCAGACGATGTATGCAC |
| FEZF1 | FEZ family zinc finger 1 | GGTACATTCCACATTCGTGAGC/ |
| | | TCACGTGCAATAATCAAAACCA |
| FGF8 | Fibroblast growth factor 8 | ACAGCGCTGCAGAATGCCAAGT/ |
| | | GAAGTGGACCTCACGCTGGTGC |
| FOXA1 | Forkhead box A1 | GGGCAGGGTGGCTCCAGGAT/ |
| | | TGCTGACCGGGACGGAGGAG |
| FOXA2 | Forkhead box A2 | CCGTTCTCCATCAACAACCT/ |
| | | GGGGTAGTGCATCACCTGTT |
| GAPDH | Glyceraldehyde-3-phosphate | TTGAGGTCAATGAAGGGGTC/ |
| | dehydrogenase | GAAGGTGAAGGTCGGAGTCA |
| GBX2 | Gastrulation brain homeobox 2 | GTTCCCGCCGTCGCTGATGAT/ |
| | | GCCGGTGTAGACGAAATGGCCG |
| GDF7 | Growth differentiation factor 7 | GACGCTGCTCAACTCCATGGCA/ |
| | | TTGGCGGCGTCGATGTAGAGGA |
| HOXA1 | Homeobox A1 | GTACGGCTACCTGGGTCAAC/ |
| | | ACTTGGGTCTCGTTGAGCTG |
| HOXA2 | Homeobox A2 | CGTCGCTCGCTGAGTGCCTG/ |
| | | TGTCGAGTGTGAAAGCGTCGAGG |
| HOXA3 | Homeobox A3 | GGCCAATCTGCTGAACCTCA/ |
| | | GAGTTCAGATAGCCACCGGC |
| FOXG1 | Forkhead box G1 | TCAACGGCATCTACGAGTTCAT/ |
| | | AAGCACTTGTTGAGGGACAGAT |
| HOXB1 | Homeobox B1 | GGCCTTCTCAGTACTACCCTCT/ |
| | | CCGTAGCTCGAGGGATGAAAAT |
| IRX3 | Iroquois homeobox 3 | GGCTTGCGCCCCGTAGAAATGT/ |
| | | AGGAGCCAGGTCAGGTCCGAAC |
| LHX2 | LIM homeobox 2 | GGGCGACCACTTCGGCATGAA/ |
| | | CGTCGGCATGGTTGAAGTGTGC |
| LMX1A | LIM homeobox transcription factor 1 | CGCATCGTTTCTTCTCCTCT/ |
| | alpha | CAGACAGACTTGGGGCTCAC |
| NKX2-1 | NK2 homeobox 1 | AGGGGGGGGCACAGATTGGA/ |
| | | GCTGGCAGAGTGTGCCCAGA |
| NKX6-1 | NK6 homeobox 1 | GGATCCCAACTCGGACGACGAGA/ |
| | | AGGATGAGCTCTCCGGCTCGG |
| OTX2 | Orthodenticle homeobox 2 | ACAAGTGGCCAATTCACTCC/ |
| | | GAGGTGGACAAGGGATCTGA |
| PAX5 | Paired box 5 | CCCCATTGTGACAGGCCGTGAC/ |
| | | TCAGCGTCGGTGCTGAGTAGCT |
| PAX6 | Paired box 6 | TGGTATTCTCTCCCCCTCCT/ |
| | | TAAGGATGTTGAACGGGCAG |
| PAX7 | Paired box 7 | CTTCAGTGGGAGGTCAGGTT/ |
| | | CAAACACAGCATCGACGG |
| PAX8 | Paired box 8 | ATAGCTGCCGACTAAGCATTGA/ |
| | | ATCCGTGCGAAGGTGCTTT |
| SHH | Sonic hedgehog | CCAATTACAACCCCGACATC/ |
| | | AGTTTCACTCCTGGCCACTG |
| SIX3 | SIX homeobox 3 | ACCGGCCTCACTCCCACACA/ |
| | | CGCTCGGTCCAATGGCCTGG |
| SIX6 | SIX homeobox 6 | CTCAACAAGAATGAGTCGGTGC/ |
| | | ACTCCTTGGTGAACTTGTGGTT |
| SOX10 | SRY-box 10 | CTTTCTTGTGCTGCATACGG/ |
| | | AGCTCAGCAAGACGCTGG |
| TBR1 | T-box, brain 1 | TCGTCCCCGCTCAAGAGCGA/ |
| | | CCTTGGCGCAGTTCTTCTCGCA |
| WNT1 | Wnt family member 1 | GAGCCACGAGTTTGGATGTT/ |
| | | TGCAGGGAGAAAGGAGAGAA |
| WNT3A | Wnt family member 3A | GCGATGGCCCCACTCGGATACT/ |
| | | TAGCTGCCCAGAGCCTGCTTCA |
| ACTB | Beta-actin | ATGTGGCCGAGGACTTTGATTG/ |
| | | ATGGCAAGGGACTTCCTGTAAC |
| PDGFC | Platelet-derived growth factor C | ACAAGGAACAGAACGGAGTACA/ |
| | | GTATGAGGAAACCTTGGGCTGT |
| ALCAM | Activated leukocyte cell adhesion | GTCTGCTCTTCTGCCTCTTGAT/ |
| | molecule | TACGTCAAGTCGGCAAGGTATG |
| APCDD1 | Adenomatosis polyposis coli down- | GGAGGGCTTTTAAGGAGTCACA/ |
| | regulated 1 | CACTGTGATCCTTGCACCATTG |
| CD47 | CD47 molecule; integrin-associated | TGGAGGCACAAAACACTACTGA/ |
| | protein | TGGGGACAGTGGACTTGTTTAG |
| CD83 | CD83 molecule | CACGGCTACAGAGTATCTTCCC/ |
| | | GGGGAGGTAACTGGGAGAAAAG |
| CNTN2 | Contactin 2 | GTCACGGGAGTACCAGAACG/ |
| | | TGTAGACAAAGTACTGGGCATCG |
| CPE | Carboxypeptidase E | CTCTGAAGACCTACTGGGAGGA/ |
| | | GCATTCGCAATTGGGTTACCTT |
| FOLR1 | Folate receptor alpha | CATTTCTACTTCCCCACACCCA/ |
| | | GGCTGTAGTTGCTGACCTTGTA |
| LGI1 | Leucine rich glioma inactivated 1 | CAACAATCTCCAGACACTCCCA/ |
| | | CCCCTCAGGTCCACATTTGTTA |
| LRTM1 | Leucine-rich repeats and | GGAAGGTGGAAGAAAAAGAGCG/ |
| | transmembrane domains 1 | GGAAACACATCAGCCCTACTCA |
| SERPINF1 | Serpin family F member 1 | TCGGACCCTAAGGCTGTTTTAC/ |
| | | CTTTCAGGGGCAGGAAGAAGAT |
| SLC39A8 | Solute carrier family 39 member 8; | GCTACCCAAATAACCAGCTCCA/ |
| | ZIP8 | CGATCCTCACATGGGTGAAAGT |
| TFF3 | Trefoil factor 3 | TCTGGAGCCTGATGTCTTAACG/ |
| | | GACGCAGCAGAAATAAAGCACA |
| TPBG | Trophoblast glycoprotein | AATAATTCGCTGGTGAGCCTGA/ |
| | | CCAGGTGGAGGCTTTCTAGATG |

### Image Acquisition and Analysis

Image acquisition was done on a Leica DMI6000 B and a PerkinElmer Operetta CLS High-Content Analysis System. For quantification of TH⁺ and HuNu⁺ cells in immunohistochemistry, Z-stack images under 20× magnification were captured and stitched automatically. For IHC, graft-derived TH⁺ cells were counted manually and HuNu⁺ cells were quantified semi-automatedly. For immunofluorescence, the number of TH, FOXA2, LMX1 cells were manually quantified.

### Statistical Analysis and Figure Illustration

Statistical tests were performed in RStudio. A critical significance level of 0.05 was used for all statistical tests. A Shapiro-Wilk test and inspection of frequency distribution histograms was used to assess normality. Gaussian distributions were analysed by using paired or unpaired t-tests. To investigate whether two or more samples of non-normal distributions were statistically significantly different, pairwise Wilcoxon-Mann-Whitney U test or a Dunn's test was used as post hoc tests following a Kruskal-Wallis test. Non-linearity was assessed, and correlations were assessed by using linear regression models.

### Results

Identifying Novel Candidate Markers of cVM Progenitors from scRNA-Seq Data

Previous publications have identified cell surface markers which label dopaminergic progenitor cells (Bye et al. 2015; Doi et al. 2014; Fathi et al. 2018; Gennet et al. 2016; Kikuchi et al. 2017; Lehnen et al. 2017; Paik et al. 2018; Samata et al. 2016; Sundberg et al. 2013; Yoo et al. 2021) (Table 3).

To identify the candidate cell surface markers which showed the highest specificity for cVM progenitor cells compared to other neural progenitor cells from other brain regions; in particular the commonly contaminating and highly similar rVM cells, which do not give rise to DA neurons, the inventors utilised a previously published microfluidics-based in vitro model of the developing neural tube, which recapitulates progressive rostro-caudal patterning of neural progenitor cells from the forebrain to the midbrain and hindbrain (Rifes et al. 2020).

To identify candidate cell surface markers of a cVM fate, the scRNA-seq dataset from the ventral microfluidic neural tube tissue were screened to find genes that were specifically expressed in the VM. By screening for VM-specific expression patterns, the novel cell surface marker APCDD1 as well as TPBG were identified as candidate markers of general VM progenitor cells. In contrast, a vMB/MHB-specific expression was not observed in previously published cell surface markers indicative of cVM progenitor cells (FIG. 1A). The expression pattern of APCDD1 was validated by immunolabelling of a 5-week human foetus, confirming a cVM-specific, surface marker-like expression (FIG. 1B). As expected, APCDD1 was not detected in a 11-week human foetal VM, implying a transient expression pattern.

Thus, scRNA-seq shows that APCDD1 and TPBG are more enriched for a VM fate compared to previously published markers, and VM-specific expression of APCDD1 was confirmed in human foetal tissue.

### Benchmarking the Regional Specificity of Published and Novel Cell Surface Markers

It was important to benchmark the regional enrichment of the newly discovered candidate markers compared to previously published surface markers. For this purpose, hESCs were patterned to day 16 (d16) progenitors of a dorsal forebrain (dFB), ventral forebrain (vFB), dorsal midbrain (dMB), rostral and caudal VM (rVM and cVM), dorsal hindbrain (dHB) and ventral hindbrain (vHB) neural progenitor fate (FIG. 3A), and qRT-PCR was performed, using a panel of rostral-caudal and dorsal-ventral markers. In line with the scRNA-seq from d14 ventral MiSTR, it was shown that APCDD1 and TPBG were the most enriched markers in the caudal VM DA fate on a transcriptional level compared to previously published surface markers (FIG. 3B, FIG. 5A-B). Of note, there was a statistically significant correlation between TPBG and CORIN, but not for APCDD1 and CORIN. Previously published markers ALCAM, CORIN, CD47/IAP, CNTN2 and LRTM1 showed enriched expression in rVM progenitor cultures with comparatively less expression in the cVM DA progenitor cells. Expression of FOLR1 was not enriched in VM cultures compared to other regional cultures, and FOLR1 did not appear as a useful marker of VM cells (FIG. 3B). In line with this finding, the mRNA expression of APCDD1 and TPBG correlated well with VM markers such as FOXA2, LMX1A and EN1 (FIG. 3C-E, FIG. 5B). By using a linear regression model, it was shown that the expression levels of APCDD1 correlated highly to LMX1A, FOXA2 and EN1 (adjusted R²=0.95, p=1.79e-19), further validating APCDD1 as a marker for a VM DA fate (FIG. 3F).

To further validate these results, a flow cytometric-based regional specificity assay was developed and performed by comparing previously published antibodies against putative cell surface markers indicative of VM progenitor cells (Bye et al. 2015; Doi et al. 2014; Fathi et al. 2018; Gennet et al. 2016; Kikuchi et al. 2017; Lehnen et al. 2017; Paik et al. 2018; Samata et al. 2016; Sundberg et al. 2013) to APCDD1. For this purpose, d16 regionalised neural progenitor cells (same batches as used for qRT-PCR) were analysed for cell surface protein marker expression by flow cytometry, using fluorophore-conjugates antibodies against each of the candidate cell surface markers (FIG. 2A-J). For markers which have been previously published, the same antibodies and similar concentrations as used in the publications were used. Two anti-LRTM1 antibodies were tested at a variety of concentrations as well as several secondary antibodies, confirming the low percentage of LRTM1⁺ cells (FIG. 2G-H).

### Determination of the Potential Influence of Dissociation Method

To assess the potential influence of the dissociation method on the results, cells from all regional fates were dissociated with either Accutase, TrypLE or Papain, and were analysed by flow cytometry. By performing a factor analysis for mixed data (FAMD), it was shown that the dissociation method was a relatively small contributor of variance observed globally (FIG. 6B). However, by using the suitable parametric or non-parametric, multiple-comparison test, it was shown that the percentage of positive cells was statistically significantly lower for Papain compared to Accutase and TrypLE for APCDD1, CORIN, and CNTN2 (FIG. 7A, FIG. 6D).

Based on the percentage of positive cells, APCDD1 and TPBG appeared the most specific for labelling a cVM progenitor fate (FIG. 7C, FIG. 4A-C). To quantify the regional enrichment, a model was devised wherein a specificity index for cVM population was determined by summarizing the percentage of positive cells for each marker in cVM versus non-cVM, grouping by dissociation method and batch. By using the non-parametric, multiple-comparison Dunn's test with Benjamini-Hochberg false discovery rate adjustment, it was shown that APCDD1 and TPBG had a statistically significantly higher specify index compared to most previously published markers, i.e., were more enriched for the cVM progenitor cells (FIG. 7D). However, the mean fraction of TPBG⁺ cells in the cVM was lower than that of APCDD1⁺ cells for Accutase- and TrypLE-dissociated cells. By using level-matched, paired t-tests, it was determined that the differences were not statistically significant however, due to the high variance of TPBG⁺ cells (FIG. 4E). Indeed, there was a statistically significantly larger batch-to-batch variation in the fraction of TPBG⁺ cells for cVM progenitors cells compared to APCDD1 (FIG. 7E). Additionally, an increased standard error in the percentage positive cVM progenitors for TPBG compared to APCDD1 was devised across dissociation methods (FIG. 4F). The findings were then expanded to other cells lines, validating that APCDD1 was enriched for cVM progenitors also in the hESC line H9 (FIG. 6C).

### Temporal Expression of APCDD1

To investigate the temporal expression of these cell surface markers, d11 cVM progenitors were analysed with the surface marker flow cytometric panel and the expression levels were compared to expression in the same batches at day 16. For Accutase-dissociated cVM cells, the percentage of APCDD1⁺ cells were lower on d11 compared to d16 and a larger spread of APCDD1 expression was found on d11 (94.3±1.6 and 43.5% APCDD1⁺±21.1 SEM, FIG. 7B, FIG. 4D). This implies that d11 is a too early time-point to use APCDD1 and TPBG for QC, since markers of authentic cVM progenitors are not significantly and robustly upregulated until d16, which corresponds to the temporal expression on EN1 (Kirkeby et al. 2017; Nolbrant et al. 2017; Rifes et al. 2020).

### Use of APCDD1 to Discriminate Between rVM and cVM Progenitors

VM progenitor cells are characterised by the co-expression of the two regional transcription factors FOXA2 and OTX2 (Arenas, Denham, and Villaescusa 2015). Therefore, the co-expression of APCDD1 with FOXA2/OTX2 double-positive cells was investigated to validate the feasibility of APCDD1 as a QC purity marker for the VM population. To achieve this, a panel was developed where extracellular labelling with an APCDD1 antibody was performed first, followed by intracellular labelling with antibodies against FOXA2 and OTX2. The percentage of APCDD1⁺ cells was highly correlated with that of FOXA2⁺/OTX2⁺ cells (adjusted r-squared=0.95, p=6.77e11, FIG. 7F-H), confirming that APCDD1 can be used as a surrogate marker for identifying the VM population. Next, to identify a potential dual cell surface marker panel which can rapidly and robustly discriminate successful batches of dopaminergic cVM progenitors from failed batches of rVM progenitor cells for PD cell therapy, different combinations of APCDD1 and TPBG were tested (enriched in the cVM population, FIG. 3B, 7C-D, 4A-C) together with CORIN (enriched in the rVM population, FIG. 3B, 4A-C). Based on this, it was shown that a dual, APCDD1⁺ CORIN⁻ panel could most clearly separate between the two VM cell populations, with a mean 57-fold cVM to rVM enrichment. APCDD1+ had a 2-fold cVM to rVM enrichment (FIG. 8E, 9D). To confirm the potential of APCDD1 to identify batches that failed to meet the QC criteria, such samples were purposely included. Both APCDD1⁺ and APCDD1⁺CORIN⁻ appeared to detect batches failing to reach the cVM QC criteria, as marked by the aberrant fractions of cells within the gate. TPBG did not seem useful in this regard (FIG. 9D, 8E). Taken together, APCDD1 is a useful inclusion and/or exclusion marker for the QC of VM DA progenitor differentiations.

### Discussion

APCDD1 and TPBG were the two markers which showed the highest specificity for cVM progenitor cells compared to all previously published cell surface markers. A higher fraction of cVM progenitors were positive for APCDD1 compared to TPBG, and the variance of positive cells was lower for APCDD1. Taken together, it can be concluded that APCDD1 is a more suitable QC marker for VM DA fate compared to all previously published markers.

It was shown that the percentage of APCDD1⁺ cells correlated highly with FOXA2⁺OTX2⁺ cells, and that APCDD1⁺ cells, particularly used with CORIN⁻ cells, discriminated between rVM and cVM progenitors with high accuracy and detected failed batches of cVM progenitors. Together, these results validate the use of APCDD1 as a marker of VM DA cell differentiations.

TABLE 3
Summary of cell surface markers used for isolating ventral midbrain dopamine
progenitors.

| | | Diff. | Results from sorting | Control | Results from in | |
|---|---|---|---|---|---|---|
| | Cell type(s) | proto- | in vitro (% + pre- vs. | popula- | vivo | |
| Marker | tested | col | post-sorting) | tion | transplantation | Reference |
| CORIN | 404C2, | (Doi | LMX1A⁺FOXA2+: | GEP | CORIN⁺ VS. | (Doi et |
| | 836B3 | et al. | 47.3 → 75.5 | CORIN⁺, | unsorted | al. 2014) |
| | hiPSCs | 2014) | | CORIN⁻, | (rat): | |
| | | | | unsorted | ↑TH⁺ cells | |
| | | | | cells | (6,750 vs. | |
| | | | | | 3,440) | |
| | N117-11, | (Doi | CORIN⁺: 19.9 → | GEP | CORIN⁺ sorted | (Kikuchi |
| | 1147F1, | et al. | 91.8 | CORIN⁺, | (primate) : | et al. |
| | 836B3, | 2014) | Ki67⁺: 7,0 | CORIN⁻, | ≥16,000 TH⁺ | 2017) |
| | 1231A3, | | | unsorted | cells | |
| | PD12-1, | | | cells | Improvement | |
| | 783E2, | | | | in PD score | |
| | 1275A3, | | | | | |
| | 1263A18 | | | | | |
| | hiPSCs | | | | | |
| LRTM1 | 440A-3 iPSC | (Doi | FOXA2⁺LMX1A⁺: | Mouse | LRTM1+ VS. | (Samata |
| | LMX1A-GFP, | et al. | 72.0 → 86.7 | HB, | unsorted | et al. |
| | EB5, G4-2 | 2014) | Ki67⁺: ~12.5 → | GEP | (rat): | 2016) |
| | mouse-ESCs | | ~5 | | ↓graft size | |
| | | | 5-HT⁺: ~12 → ~6 | | ↑TH⁺ (29.0 | |
| | | | | | vs. 4.2%) | |
| | | | | | LRTM1⁺ VS. | |
| | | | | | unsorted | |
| | | | | | (primate): | |
| | | | | | TH⁺/FOXA2⁺: | |
| | | | | | most cells | |
| | | | | | Ki67⁺/FOXA2⁺: | |
| | | | | | <0.5% | |
| IAP | hFFF-hiPSCs | (Kirke | ↑LMX1A/FOXA2/ | Roof | IAP⁺ VS. | (Lehnen |
| | SA001, H9 | by et | CORIN RNA expr. | plate-, | unsorted: | et al. |
| | hESCs | al. | | alar | ↓graft size | 2017) |
| | | 2012) | | plate- | ↑TH density | |
| | | with | | and | (TH⁺ | |
| | | modifi- | | basal | cells/mm³) | |
| | | cations | | plate | ↓ 5-HT⁺ cells | |
| | | | | progen- | (~8, ~23%) | |
| | | | | itors | | |
| FOLR1 | E14TG2A, | (Jaeger | FOLR1⁺ vs. FOLR1- | Cortical | N/A | (Gennet |
| | PITX3-GFP, | et | (post): | gluta- | | et al. |
| | LMX1A-GFP | al. | >>FOXA2⁺LMX1A⁺ | matergic | | 2016) |
| | mESCs | 2011) | cells | neurons | | |
| | H7 hESCs | | < GABA⁺, 5-HT⁺, | | | |
| | | | GFAP⁺ | | | |
| | | | DA progenitors vs. | | | |
| | | | neg. population | | | |
| | | | ↑LMX1A RNA | | | |
| | | | expr. | | | |
| ALCAM | VM from | N/A | LMX1A and NGN2 | Rat | ALCAM⁺ VS. | (Bye et |
| | GFP⁺ | | transgenic mice: | lateral | ALCAM: | al. 2015) |
| | embryos of | | ALCAM⁺: 20.2 → | MB | ~30-fold | |
| | NGN2-GFP, | | 96.2 | | ↑TH⁺ cells | |
| | LMX1A-GFP | | | | ↑TH⁺ fiber | |
| | knock-in | | | | density | |
| | mice | | | | | |
| | BJ-RiPS, | Adapted | FOXA2⁺: 59 → 82 | GEP | N/A | (Paik et |
| | 18a, 1016A | (Kirke | (Paik et al. 2018) | LMX1⁻/ | | al. 2018) |
| | and 15b | by et | | FOXA2⁻ | | |
| | hiPSCs | al. | | FB | | |
| | (Paik et al. | 2012; | | progen- | | |
| | 2018) | Kriks | | itors vs. | | |
| | | et al. | | LMX1⁺/ | | |
| | | 2011) | | FOXA2⁺ | | |
| | | | | DA | | |
| | | | | progen- | | |
| | | | | itors | | |
| | | | | (Paik et | | |
| | | | | al. | | |
| | | | | 2018) | | |
| CNTN2 | LMX1A-GFP | (Kirke | CNTN2⁺: 19.9 → | Rostral | CNTN2⁺ VS. | (Fathi et |
| | H6, H9 | by et | ~60 | FB | unsorted: | al. 2018) |
| | hESCs | al. | LMX1A⁺: ~45 → | progen- | ↑ forelimb | |
| | | 2012) | 84 | itors, | akinesia | |
| | | | FOXA2⁺: ~65 → | caudal | ↑TH⁺ (~13, | |
| | | | 91 | spinal | ~8%) | |
| | | | | progen- | | |
| | | | | itors | | |
| NCAM⁺/CD29^{low} | hESCs: H9; | (Denham | FOXA2⁺/TH⁺: | Unsorted | NCAM+/CD29^{low} | (Sundberg |
| | hiPSCs: | et | >10 → ≤40 | cells | vs. unsorted: | et al. |
| | 2135 and | al. | SSEA-4⁺: a few | | ↑TH⁺ cells | 2013) |
| | 1815 hiPSCs | 2012; | cells (pre) > no | | motor | |
| | MF95.06, | Kriks | cells | | phenotype | |
| | MF27.04, | et al. | | | rescued | |
| | MF66.02, | 2011) | | | according to | |
| | MF25.04 | | | | net turns/min | |
| | primate- | | | | | |
| | iPSCs | | | | | |
| TPBG | H9 hESCs, | (Kirke | Day 20 | Vehicle, | TPBG⁺ VS. | (Yoo et |
| | LMX1A-eGFP | by et | ↑LMX1A RNA | Unsorted | TPBG⁻ and | al. 2021) |
| | hESCs | al. | expr. | cells, | unsorted cells | |
| | | 2012) | Day 52 | eGFP⁺, | ↑TH⁺ cells | |
| | | with | TH⁺: ~45 → ~65 | eGFP⁻, | (~35%) | |
| | | modifi- | PITX3⁺: ~15 → | TPBG⁺, | graft | |
| | | cations | ~25 | TPBG⁻ | volume | |
| | | | | | Improved | |
| | | | | | motor deficits | |

### Example 2

### Background

As described in Example 1, caudal ventral midbrain (cVM) progenitor cells can be identified by measuring the presence and/or amount of APCDD1 expressed in the cells. This method can be further used to purify a population of cVM progenitor cells by isolating from a group of cells the cells that express APCDD1 to an amount that identifies them as cVM cells.

The inventors evaluated the potential to isolate cVM cells based on the expression of APCDD1 from mixed populations of cells and assessed the levels of cVM progenitor cell enrichment and of non-cVM progenitor cell contamination.

### Materials and Methods

### Fluorescence-Activated Cell Sorting (FACS)

For FACS, cryopreserved progenitors, which had been patterned toward neuronal fates, were thawed, and resuspended in Wash buffer (0.5% serum, balanced salt solution). The cells were centrifuged and were then resuspended in Staining buffer (2.5% serum, balanced salt solution) to a final density of 1e6 cells/mL. The cells were incubated with primary antibodies on ice for 30 min. The cells were washed three times by the addition of Wash buffer, followed by centrifugation. Secondary antibodies were added to cells labelled with an indirectly conjugated antibody, and the cells were incubated as above. The washing step was thereafter repeated. Finally, the cells were resuspended in Wash buffer+viability dye (1:1,000) and were thereafter analysed and sorted on a FACS sorter. Gates were set based upon fluorescence minus one (FMO)-controls and the compensation was determined by using single-stained and an unstained control. Post-processing, QC, and analysis of collected data was done by using the software program FlowJo (version 10.7.2). A list of reagents and dilutions used for flow cytometry and FACS can be found in Table 1.

### Results

To evaluate the value of APCDD1 for predicting dopaminergic cell fate, APCDD1⁺ and APCDD1⁻ cells were sorted from high-purity batches of cVM progenitor cells as well as STEM-PD GMP3 (batch of cells meant to be used in the STEM-PD phase-I clinical trial) and STEM-PD GMP5 (FIG. 8A-B). qRT-PCR analysis revealed that sorting for APCDD1⁺ cells enriched for genes indicative of a cVM fate, such as EN1, FOXA1, LMX1A and SHH, while genes marking contaminating cell populations (i.e., NKX2-1, ISL1 and PITX2) were enriched in the APCDD1⁻ population (FIG. 8C). To assess the maturation capacity of the cells sorted on day 16, APCDD1⁺ and APCDD1⁻ cells were further seeded for terminal maturation in vitro, and only the APCDD1⁺ cultures were rich in TH⁺ neurons with stereotypic neuronal morphology after 42 days in vitro (FIG. 8D).

To test whether sorting for APCDD1⁺ cells could rescue an incorrectly patterned cVM batch, cVM, rVM and vHB-patterned progenitor cells were combined to yield approximately 50% APCDD1⁺ cells, based upon previous flow cytometric analysis of the cells. The cells were thereafter sorted for APCDD1⁺ and APCDD1⁻ cells (FIG. 10A-B). Many SOX1⁺ and ISL1/2⁺ cells were observed by ICC in the APCDD1⁻-sorted cells at 18 days in vitro. Conversely, the APCDD1⁺-derived cells were almost devoid of SOX1⁺ cells and completely depleted of ISL1/2⁺ cells at the same time-point. Analogous to the results from sorting of pure batches of cVM progenitor, a statistically significant enrichment was observed for the expression levels of genes indicative of a VM DA fate in the APCDD1⁺ fraction while contaminating, non-VM DA genes (if expressed) were depleted (FIG. 10C).

### Example 3

As described in Examples 1 and 2, caudal ventral midbrain (cVM) progenitor cells can be identified by measuring the presence and/or amount of APCDD1 expressed in the cells and can be further used to purify a population of cVM progenitor cells by isolating from a group of cells the cells that express APCDD1 to an amount that identifies them as cVM progenitor cells. The isolated cVM progenitor cells can further be used in cell replacement therapies.

### Materials and Methods

### Animal Experiments

All procedures were conducted in accordance with the European Union Directive (2010/63/EU) and was approved by the local ethical committee at Lund University as well as the Swedish Department for Agriculture (Jordbruksverket). Adult, female, athymic nude rats (Hsd:RH-Foxn1^{rnu}) were purchased from Envigo and were housed on a 12:12-hr light:dark cycle with ad libitum access to food and water. For all surgical procedures, rats (>225 g) were anesthetized via intraperitoneal injection of a 20:1 or 3:2 mixture of fentanyl-dormitor or ketaminol-dormitor (Apoteksbolaget), respectively, according to their weight. Rats were unilaterally lesioned by intracranial injection of 10.5 μg 6-hydroxydopamine to the medial forebrain bundle. The extent of the lesion was assessed by amphetamine-induced rotations test. For this purpose, 3.5 mg/kg amphetamine was administered by intraperitoneal injection, and median net turns per min were determined over a duration of 90 min. Cryopreserved RC17 hESCs were prepared as previously described (Kirkeby et al. 2012; Nolbrant et al. 2017). 200,000-240,000 cells were injected, depending on the yield from the FACS, as described previously (Tiklová et al. 2020). Nude rats were injected at the striatal coordinates AP, +0.9/+1.4; ML, −3.0/−2.6; DV, −5.0/−4.0. SD rats were administered 10 mg/kg cyclosporine by daily intraperitoneal injections two days prior to transplantation, and until euthanisation, 18 weeks post-transplantation.

### Tissue Handling

Rat brains were fixed in 4% (wt/vol) paraformaldehyde by perfusion, according to standard protocol. The brains were removed, and post-fixed over-night before being dehydrated in 25% (wt/vol) sucrose. Brains were sectioned with a thickness of 35 μm in 1:8 series by using a freezing microtome (Leica), and were stored in Buffer G (13 mM NaH₂PO₄, 38 mM Na₂HPO₄ 30% (vol/vol) ethylene glycol, 30% (vol/vol) glycerol) at −20° C. Foetal tissue was sectioned 12 μm thick, slides stored at −20° C.

### Immunohistochemistry (IHC)

IHC was performed on free-floating sections, which for most steps were placed mesh wells in 12-well plates. Between each step, sections were washed for 3×5 min in PBS. For antigen retrieval, sections were incubated in Buffer H (10 mM Tris Base, 1 mM EDTA Solution, 0.05% Tween 20, pH 9.0, H₂O), followed by incubation at 80° C. Quenching was performed by incubation in Buffer I (10% (vol/vol) methanol, 3% (vol/vol) H₂O₂, PBS). Sections were thereafter blocked in Buffer J (0.244% vol/vol Triton-X, 5% (vol/vol) species-specific serum, PBS). For certain markers, the sections were then blocked by 2×15 min incubation in avidin/biotin solutions according to the manufacturer's instructions (Vector laboratories). The sections were then incubated with the serum-diluted, primary antibody over-night. Thereafter, the sections were incubated in the serum for 15 min, followed by incubation in the biotinylated secondary antibody for 1 h. Sections were then incubated in ABC horseradish peroxidase complex (Vector laboratories) for 1 h, prepared as instructed (Vector laboratories). Subsequently, sections were incubated in 0.5 mg/mL DAB solution+0.125% (vol/vol) H₂O₂, or in the DAB substrate kit solution (Vector laboratories) for a suitable time (1-3 min). To visualise the histology due to high signal:noise ratio, DAB/nickel-labelled sections were counter-stained with Gill II haematoxylin for 1 min and were thereafter washed for 2×1 min. Sections were mounted, dehydrated and coverslipped according to standard protocol with the appropriate mounting medium. A list of reagents and dilutions used for IHC and ICC can be found in Table 1.

### Immunofluorescence

Immunofluorescent labelling on rat tissue was performed similarly as for IHC but excluding quenching and avidin/biotin blocking. Slides with foetal tissue was thawed to room-temperature and were then briefly immersed in PBS. The sections were blocked for 30 min in Buffer J, and were thereafter incubated in serum-diluted, primary antibodies over-night at 4° C. The sections were then washed in PBS for 30 min. Thereafter, the sections were incubated in serum-diluted, secondary antibodies (1:500) for 30 min. Subsequently, the sections were washed for 2×30 min in PBS. Sections were coverslipped with FluorSave™ (Sigma Aldrich).

### Image Acquisition

For quantification of TH⁺ and HuNu⁺ cells in DAB-developed IHC sections, brightfield, 5 Z-stack images (~3.0 μm apart) under 20× magnification were captured and stitched automatically by using a Leica DMI6000B with the LAS-X software program. ICC images were acquired on the same microscope, and immunofluorescent images on an Operetta CLS High-Content Analysis System (PerkinElmer) with a 20× confocal objective in 6 Z-stacks (~3.4 μm apart).

### Manual and Semi-Automated Image Analysis

The number of DAB-labelled TH⁺ cells as well as immunofluorescently labelled TH⁺, LMX1A⁺ and FOXA2⁺ single-, double and triple-positive cells were counted manually in the software program Fiji (version 2.1.0). HuNu⁺ cells were counted semi-automatedly by using the same program. For this purpose, the background was subtracted, Otsu's method binarized the image (Smith et al. 1979), and particles were then segmented by using the built-in watershed algorithm. The number of cells was quantified by using the ‘Analyze Particles’ option with 0.05-infinity as particle size. The yield of TH⁺ cells per animal is shown as the total number of TH⁺ cells divided by the total number of HuNu⁺ cells, or as estimated total number of TH⁺ cells per 1e5 cells transplanted.

### Convolutional Neuronal Network-Based Automated Image Analysis Pipeline—SIMPLIcity

A multiplexed image analysis pipeline was developed built upon the SIMPLI (Bortolomeazzi et al. 2022) pipeline to perform pre-processing, single-cell data extraction (segmentation) with StarDist (Schmidt et al. 2018), and downstream analysis. The pipeline was named SIMPLIcity.

To reshape the exported images from Operetta Harmony software to enable downstream analysis, they were processed by using PerkinElmer Operetta Stitcher (4.1) in Fiji. The stacks were projected with the standard deviation projection method and were exported as single-channel images. The channels were processed and analyzed independently after manually cut to 512×512 or 1024×1024 patches. The original CellProfiler4 (Stirling et al. 2021) pre-processing was replaced. Instead, for brightfield images, we utilized SIMPLI's built-in pixel normalization, followed by median filtering to remove salt-and-pepper noise, and non-local means denoising algorithm to remove Gaussian noise. Finally, the image contrast was enhanced with contrast limited adaptive histogram equalization. For images acquired on Operetta CLS, pre-processing was performed by normalizing pixel values while maintaining the image quality. In addition, the pre-processing scripts performed image thresholding with a user-specified thresholding algorithm. To increase the segmentation accuracy, models were trained with several markers. To create corresponding, binary masks, images were annotated manually by using Labkit (v. 0.3.5) (Arzt et al. 2022) plugin in Fiji. To increase the size of training sets, an additional dataset from StarDist GitHub page was included for the training of HuNu- and LMX1A-models. The full dataset consisted of 497 single-channel images. To achieve a stable learning process and optimal model performance, the learning rate, batch size, number of epochs, and dropout were adjusted. The default loss function in StarDist called means absolute error was used to ensure an accurate learning process. Scripts provided by StarDist were used for the training. The HuNu and LMX1A models were trained on HPC2N's Kebnekaise supercomputer using Intel Xeon E7-8860v4 GPU. After well performing models were yielded, transfer learning from the ZeroCostDL4Mic platform (von Chamier et al. 2021), which is compatible with StarDist networks, was used to train the remaining markers. A comprehensive LMX1A-TH model was trained to segment both LMX1A and TH channels.

To increase the accuracy of the image segmentation, each channel was segmented separately, instead of merging the images prior to segmentation. A slightly modified version of the original Nextflow pipeline was used. To ensure an inter-operating system compatible pipeline, the Singularity containers were replaced with Docker containers that were hosted on Docker Hub, which manages dependencies. SIMPLIcity can be used as a command line and web-based tool. The dependencies in the web app are managed with a Conda environment.

### Results

Sorting for APCDD1 Generated DA-Rich Grafts after Transplantation and Depletes Contaminating Cell Populations

To validate the predictive value of APCDD1 on in vivo outcomes cVM, rVM and vHB-patterned progenitors were combined as described above. APCDD1⁺ and APCDD1⁻ cells were thereafter sorted, which were then transplanted to Parkinsonian nude rats (FIG. 11A). 28-weeks post-transplantation, animals injected with APCDD1⁺ cells had full behavioural rescue, determined by using amphetamine-induced rotations test, whereas no improvements were observed with rats transplanted with APCDD1⁻ cells. (FIG. 11H). The grafts derived from APCDD1⁺ cells had statistically significantly more TH⁺ cells per transplanted cells compared to APCDD1⁻ grafts (2233±532 SEM vs. 292±43 total TH⁺ cells per rat, p=0.018), corresponding to a mean increase of 664% TH⁺ cells (FIG. 11F). In line with this finding, there were more TH⁺ neurons per human nuclear antigen (HuNu)⁺ cells (FIG. 11G). However, in the APCDD1⁻-derived grafts, many high-expressing TH⁺ cells had a non-A9 DA neuron-like morphology (FIG. 11D′-E″). Based upon this, it can be hypothesized that the TH⁺ cells in the APCDD1-sorted groups belonged to biologically separate subtypes of TH neurons, and that at least a subset of the TH⁺ cells in the APCDD1⁻-derived grafts were not bona fide VM DA neurons. To confirm this, an image analysis pipeline based upon SIMPLI (Bortolomeazzi et al. 2022) was developed, as the pre-trained models failed to accurately segment objects. By training models and thereafter using transfer learning, TH⁺ cells could accurately be identified. After segmentation, the frequency distribution of the pixel intensity values was compared. It was shown that a subset of the TH⁺ cells in the APCDD1⁻-derived grafts had a statistically significant increased TH pixel intensity compared to the APCDD1⁺-derived grafts. Indeed, APCDD1⁺-derived grafts were devoid of this subset of high-expressing TH⁺ cells, implying that a subset of the TH⁺ cells are intrinsically different between the APCDD1-sorted groups. Further characterization of the graft composition was done by determining the fraction of cells co-expressing TH/FOXA2/LMX1A. The APCDD1⁺-derived grafts had a statistically significantly increased ratio of TH/LMX1A/FOXA2 triple-positive cells (p=0.033, FIG. 12C).

In summary, although both APCDD1-sorted groups produced TH⁺ neurons after transplantation to Parkinsonian rats, only APCDD1⁺ cells were predictive of functional VM DA neurons, whereas APCDD1⁻ cells produced non-A9-like DA neurons that provided no amelioration of the behavioural deficits.

Taken together, Examples 1-3 herein show that APCDD1 is a novel marker of cVM progenitor cells, and has utility in quality control and purification of cVM progenitors for use in clinical trials and therapeutics. APCDD1 is highly specific to cVM progenitors and can therefore be used to isolate these cells with higher reproducibility and purity than previously identified markers.

### REFERENCES

Arenas, Ernest, Mark Denham, and J. Carlos Villaescusa. 2015. “How to Make a Midbrain Dopaminergic Neuron.” Development (Cambridge) 142(11): 1918-36.
Arzt, Matthias et al. 2022. “LABKIT: Labeling and Segmentation Toolkit for Big Image Data.” Frontiers in Computer Science 4(February): 1-12.
Barker, Roger A. et al. 2019. “Designing Stem-Cell-Based Dopamine Cell Replacement Trials for Parkinson's Disease.” Nature Medicine 25 (July 2019).
Björklund, A. et al. 1980. “Reinnervation of the Denervated Striatum by Substantia Nigra Transplants: Functional Consequences as Revealed by Pharmacological and Sensorimotor Testing.” Brain Research 199(2): 307-33.
Bolam, J P et al. 1987. “Synaptic Input and Local Output of Dopaminergic Neurons in Grafts That Functionally Reinnervate the Host Neostriatum.” Exp Brain Res 68(1): 131-46.
Bortolomeazzi, Michele et al. 2022. “A SIMPLI (Single-Cell Identification from MultiPLexed Images) Approach for Spatially-Resolved Tissue Phenotyping at Single-Cell Resolution.” Nature Communications 13(1).
Bye, Chris R. et al. 2015. “Transcriptome Analysis Reveals Transmembrane Targets on Transplantable Midbrain Dopamine Progenitors.” Proceedings of the National Academy of Sciences of the United States of America 112(15): E1946-55.
von Chamier, Lucas et al. 2021. “Democratising Deep Learning for Microscopy with ZeroCostDL4Mic.” Nature Communications 12(1): 1-18. http://dx.doi.org/10.1038/s41467-021-22518-0.
Connolly, Barbara S., and Anthony E. Lang. 2014. “Pharmacological Treatment of Parkinson Disease: A Review.” JAMA—Journal of the American Medical Association 311(16): 1670-83.
Crameri, Fabio, Grace E. Shephard, and Philip J. Heron. 2020. “The Misuse of Colour in Science Communication.” Nature Communications 11(1): 1-10. http://dx.doi.org/10.1038/s41467-020-19160-7.
Denham, Mark et al. 2012. “Glycogen Synthase Kinase 3P and Activin/Nodal Inhibition in Human Embryonic Stem Cells Induces a Pre-Neuroepithelial State That Is Required for Specification to a Floor Plate Cell Lineage.” Stem Cells 30(11): 2400-2411.
Doi, Daisuke et al. 2014. “Isolation of Human Induced Pluripotent Stem Cell-Derived Dopaminergic Progenitors by Cell Sorting for Successful Transplantation.” Stem Cell Reports 2(3): 337-50. http://dx.doi.org/10.1016/j.stemcr.2014.01.013.
Dunnett, S. B. et al. 1983. “Intracerebral Grafting of Neuronal Cell Suspensions. IV. Behavioural Recovery in Rats with Unilateral Implants of Nigral Cell Suspensions in Different Forebrain Sites.” Acta Physiol. Scand. 522: 29-38.
Fathi, Ali et al. 2018. “Discovery of Novel Cell Surface Markers for Purification of Embryonic Dopamine Progenitors for Transplantation in Parkinson's Disease Animal Models.” Molecular and Cellular Proteomics 17(9): 1670-84.
Freund, T. F. et al. 1985. “Efferent Synaptic Connections of Grafted Dopaminergic Neurons Reinnervating the Host Neostriatum: A Tyrosine Hydroxylase Immunocytochemical Study.” Journal of Neuroscience 5(3): 603-16.
Gennet, Nicole, Claudia Tamburini, Xinsheng Nan, and Meng Li. 2016. “FolR1: A Novel Cell Surface Marker for Isolating Midbrain Dopamine Neural Progenitors and Nascent Dopamine Neurons.” Scientific Reports 6(8): 1-10.
He, Shuai, and Shilei Tang. 2020. “WNT/@-Catenin Signaling in the Development of Liver Cancers.” Biomedicine and Pharmacotherapy 132(September): 110851. https://doi.org/10.1016/j.biopha.2020.110851.
Hochberg, Yosef. 2016. “Controlling the False Discovery Rate: A Practical and Powerful Approach to Multiple Testing Author(s): Yoav Benjamini and Yosef Hochberg Source: Journal of the Royal Statistical Society. Series B (Methodological), Vol. 57, No. 1 (1995), Publi.” 57(1): 289-300.
Jaeger, Ines et al. 2011. “Temporally Controlled Modulation of FGF/ERK Signaling Directs Midbrain Dopaminergic Neural Progenitor Fate in Mouse and Human Pluripotent Stem Cells.” Development 138(20): 4363-74.
Kagermeier-Schenk, Birgit et al. 2011. “Waif1/5T4 Inhibits Wnt/β-Catenin Signaling and Activates Noncanonical Wnt Pathways by Modifying LRP6 Subcellular Localization.” Developmental Cell 21(6): 1129-43.
Kee, Nigel et al. 2017. “Single-Cell Analysis Reveals a Close Relationship between Differentiating Dopamine and Subthalamic Nucleus Neuronal Lineages.” Cell Stem Cell 20(1): 29-40.
Kefalopoulou, Zinovia et al. 2014. “Long-Term Clinical Outcome of Fetal Cell Transplantation for Parkinson Disease: Two Case Reports.” JAMA Neurology 71(1): 83-87.
Kikuchi, Tetsuhiro et al. 2017. “Human IPS Cell-Derived Dopaminergic Neurons Function in a Primate Parkinson's Disease Model.” Nature 548(7669): 592-96. http://dx.doi.org/10.1038/nature23664.
Kirkeby, Agnete et al. 2012. “Generation of Regionally Specified Neural Progenitors and Functional Neurons from Human Embryonic Stem Cells under Defined Conditions.” Cell Reports 1(6): 703-14. http://dx.doi.org/10.1016/j.celrep.2012.04.009.
Kirkeby, Agnete et al. 2017. “Predictive Markers Guide Differentiation to Improve Graft Outcome in Clinical Translation of hESC-Based Therapy for Parkinson's Disease.” Cell Stem Cell 20(1): 135-48.
Kriks, Sonja et al. 2011. “Dopamine Neurons Derived from Human ES Cells Efficiently Engraft in Animal Models of Parkinson's Disease.” Nature 488(7378): 547-51.
Kurowska, Zuzanna et al. 2011. “Signs of Degeneration in 12-22-Year Old Grafts of Mesencephalic Dopamine Neurons in Patients with Parkinson's Disease.” Journal of Parkinson's Disease 1(1): 83-92.
Lehnen, Daniela et al. 2017. “IAP-Based Cell Sorting Results in Homogeneous Transplantable Dopaminergic Precursor Cells Derived from Human Pluripotent Stem Cells.” Stem Cell Reports 9(4): 1207-20.
Li, Jia Yi et al. 2008. “Lewy Bodies in Grafted Neurons in Subjects with Parkinson's Disease Suggest Host-to-Graft Disease Propagation.” Nature Medicine 14(5): 501-3.
Li, Wen et al. 2016. “Extensive Graft-Derived Dopaminergic Innervation Is Maintained 24 Years after Transplantation in the Degenerating Parkinsonian Brain.” Proceedings of the National Academy of Sciences of the United States of America 113(23): 6544-49.
Nolbrant, Sara, Andreas Heuer, Malin Parmar, and Agnete Kirkeby. 2017. “Generation of High-Purity Human Ventral Midbrain Dopaminergic Progenitors for in Vitro Maturation and Intracerebral Transplantation.” Nature Protocols 12(9): 1962-79. http://dx.doi.org/10.1038/nprot.2017.078.
Normile, Dennis. 2018. “First-of-Its-Kind Clinical Trial Will Use Reprogrammed Adult Stem Cells to Treat Parkinson's.” Science. https://www.sciencemag.org/news/2018/07/first-its-kind-clinical-trial-will-use-reprogrammed-adult-stem-cells-treat-parkinson-s.
Paik, Elizabeth J. et al. 2018. “Using Intracellular Markers to Identify a Novel Set of Surface Markers for Live Cell Purification from a Heterogeneous HIPSC Culture.” Scientific Reports 8(1): 1-8.
Rifes, Pedro et al. 2020. “Modeling Neural Tube Development by Differentiation of Human Embryonic Stem Cells in a Microfluidic WNT Gradient.” Nature Biotechnology (38): 1265-1273.
Samata, Bumpei et al. 2016. “Purification of Functional Human ES and IPSC-Derived Midbrain Dopaminergic Progenitors Using LRTM1.” Nature Communications 7(13097): 1-11. http://dx.doi.org/10.1038/ncomms13097.
Schmidt, Uwe, Martin Weigert, Coleman Broaddus, and Gene Myers. 2018. “Cell Detection with Star-Convex Polygons.” Lecture Notes in Computer Science (including subseries Lecture Notes in Artificial Intelligence and Lecture Notes in Bioinformatics) 11071 LNCS: 265-73.
Shimomura, Yutaka et al. 2010. “APCDD1 Is a Novel Wnt Inhibitor Mutated in Hereditary Hypotrichosis Simplex.” Nature 464(7291): 1043-47.
Smith, P et al. 1979. “NOBUYUKI OTSU.—1979—A Threshold Selection Method from Gray-Level Histograms.” IEEE Transaction on Systems, Man and Cybernetics 20(1): 62-66.
Stirling, David R. et al. 2021. “CellProfiler 4: Improvements in Speed, Utility and Usability.” BMC Bioinformatics 22(1): 1-11. https://doi.org/10.1186/s12859-021-04344-9.
Strecker, R. E. et al. 1987. “Autoregulation of Dopamine Release and Metabolism by Intrastriatal Nigral Grafts as Revealed by Intracerebral Dialysis.” Neuroscience 22(1): 169-78.
Sundberg, Maria et al. 2013. “Improved Cell Therapy Protocols for Parkinson's Disease Based on Differentiation Efficiency and Safety of HESC-, HiPSC-, and Non-Human Primate IPSC-Derived Dopaminergic Neurons.” Stem Cells 31(8): 1548-62.
Takahashi, June 2017. “Strategies for Bringing Stem Cell-Derived Dopamine Neurons to the Clinic: The Kyoto Trial.” Functional Neural Transplantation IV: Translation to Clinical Application, Part A 230: 213-26. http://dx.doi.org/10.1016/bs.pbr.2016.11.004.
Tiklová, Katarína et al. 2020. “Single Cell Transcriptomics Identifies Stem Cell-Derived Graft Composition in a Model of Parkinson's Disease.” Nature Communications 11(1). http://dx.doi.org/10.1038/s41467-020-16225-5.
Yoo, Jeong Eun et al. 2021. “Trophoblast Glycoprotein Is a Marker for Efficient Sorting of Ventral Mesencephalic Dopaminergic Precursors Derived from Human Pluripotent Stem Cells.” npj Parkinson's Disease 7(1).
Zimmerli, Dario et al. 2020. “TBX3 Acts as Tissue-Specific Component of the Wnt/β-Catenin Enhanceosome.” eLife: 1-17.

1. A method of identifying one or more caudal ventral midbrain (cVM) progenitor cells comprising or consisting of the steps of:
(i) providing one or more cells;
(ii) measuring the presence and/or amount of adenomatosis polyposis coli down-regulated 1 (APCDD1) expressed by the one or more cells;
wherein the presence and/or amount of APCDD1 expressed by the one or more cells is indicative of the one or more cells being cVM progenitor cells.

2. The method of claim 1, wherein the method is for enriching a population of cVM progenitor cells, and wherein the method further comprises:
(iii) isolating the one or more cells that are identified as cVM progenitor cells to obtain a cVM progenitor cell or population enriched for cVM progenitor cells.

3. The method of claim 1, wherein the one or more cells provided in step (i) are neural progenitor cells, optionally wherein the one or more cells provided in step (i) are neural progenitor cells that have been obtained from fetal tissue or differentiated from stem cells, optionally wherein the stem cells are induced pluripotent stem cells (iPSC) or embryonic stem cells (ES).

4. The method of claim 1, wherein the method is for quality control during preparation of cells for cell replacement therapy; and/or wherein the method is for providing cVM progenitor cells for cell replacement therapy; and/or wherein the method is for determining the suitability of cells for use in treatment, optionally wherein the treatment is cell replacement therapy.

5. The method of claim 1, wherein measuring the presence and/or amount of APCDD1 is performed using a first binding moiety specific for APCDD1, optionally wherein the measurement is performed using a second binding moiety specific for the first binding moiety; and/or wherein the first and/or second binding moiety is an antibody or antigen binding fragment thereof; and/or wherein the first and/or second binding moiety is fluorescently labelled.

6. The method of claim 1, wherein the measurement of the presence and/or amount of APCDD1 is performed by flow cytometry, qRT-PCR, RNA sequencing or other RNA-detecting methods, immunostaining, chromatography, optionally wherein measuring the presence and/or amount of APCDD1 and/or isolation of cells expressing APCDD1 is performed by flow cytometry, optionally wherein the flow cytometry is Fluorescence Activated Cell Sorting (FACS) or Magnetic Affinity Cell Sorting (MACS).

7. The method of claim 1, wherein the method is for the treatment of a neurological disease or condition, optionally wherein the neurological disease or condition is Parkinson's disease.

8. The method of claim 1, wherein the cVM progenitor cells identified by the method are capable of differentiating into ventral midbrain dopaminergic neurons.

9. The method of claim 1, wherein the cVM progenitor cells identified by the method also express one or more of the following markers: FOXA2; OTX2; LMX1A and EN1; and/or wherein the cVM progenitor cells identified by the method do not express one or more of the following markers: CORIN and CNTN2.

10. The method of claim 1, wherein the cVM progenitor cells also express FOXA2 and/or OTX2, and the method further comprises: measuring the presence and/or amount of FOXA2 and/or OTX2 expression in the one or more cells; and wherein the presence and/or amount of APCDD1 and FOXA2 and/or OTX2 on or in the one or more cells is indicative of the one or more cells being cVM progenitor cells; and/or wherein the cVM progenitor cells do not express CORIN, and the method further comprises: measuring the presence and/or amount of CORIN on the surface of the one or more cells; and wherein the presence and/or amount of APCDD1 expression on the one or more cells and the absence and/or amount of CORIN expression on the one or more cells is indicative of the one or more cells being cVM progenitor cells.

11. The method of claim 1, wherein the cVM progenitor cells have one of the following marker profiles:
(a) APCDD1 positive and CORIN negative;
(b) APCDD1 positive, FOXA2 positive, and OTX2 positive;
(c) APCDD1 positive, CORIN negative, FOXA2 positive, and OTX2 positive.

12. The method of claim 1, wherein the method is capable of discriminating between cVM progenitor cells and neural progenitor cells or between cVM progenitor cells and neural cells of one or more of the following lineages: dorsal forebrain (dFB), ventral forebrain (vFB), dorsal midbrain (dMB), rostral ventral midbrain (rVM), dorsal hindbrain (dHB) and ventral hindbrain (vHB), preferably wherein the method is capable of discriminating between cVM progenitor cells and rVM progenitor cells or rVM cells.

13. A method of treating a neurological condition or disease in a subject in need thereof comprising,
(a) identifying a cVM progenitor cell or population thereof;
(b) carrying out the method of claim 1; and
(c) administering a therapeutically effective amount of the cVM progenitor cell or population thereof to the subject,
optionally administering the cVM progenitor cell or population thereof into the brain of the subject;
optionally wherein the neurological condition or disease is Parkinson's disease.

14. A method for identifying and/or enriching for cVM progenitor cells from a population comprising one or more cells, wherein APCDD1 is a biomarker.

15. A method for identifying and/or enriching for cVM progenitor cells from a population of one or more cells comprising a binding moiety specific for APCDD1.
